(19) United States
(12) Patent Application Publication
(10) Pub. No.: US 2025/0066365 A1
(43) Pub. Date: Feb. 27, 2025

(54) Ribonucleoside Analogues Against -Sars-Cov-2

(71) Applicant: Katholieke Universiteit Leuven, Leuven (BE)

(72) Inventors: Piet Herdewijn, Leuven (BE); Zihua Zheng, Jingmen City, Hubei (CN); Elisabetta Groaz, Leuven (BE)

(21) Appl. No.: 18/694,747

(22) PCT Filed: Sep. 23, 2022
(86) PCT No.: PCT/EP2022/076521

§ 371 Date: Mar. 22, 2024

(30) Foreign Application Priority Data
Sep. 23, 2021 (EP) 21198583.3

(51) Int. Cl. C07D 487/04; A61K 31/53; B01J 31/22; B01J 31/24

(52) U.S. Cl. CPC C07D 487/04; A61K 31/53; B01J 31/2208; B01J 31/2409; B01J 2231/4211; B01J 2231/4261; B01J 2531/004; B01J 2531/824

(57) ABSTRACT

The invention relates to nucleoside derivatives of formula (I), wherein R has the same meaning as that defined in the claims and the description. The present invention also relates to pharmaceutical compositions comprising such compounds and to uses of such compounds and compositions for the treatment or prevention of viral infections, more in particular infections caused by RNA virus, such as coronavirus infections.

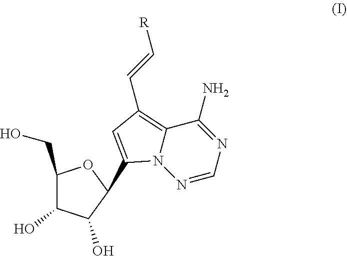

### FIELD OF THE INVENTION

The invention relates to the treatment and prevention of viral infections, in particular RNA viral infections. The invention relates to the synthesis of novel 7-styryl-pyrrolo[2,1-f][1,2,4]triazine C-ribonucleoside analogues and their use against viral infections, in particular infections caused by coronavirus viruses such as SARS-CoV-2.

### BACKGROUND OF THE INVENTION

COVID-19 is an acute respiratory disease caused by SARS-CoV-2 which is a positive-sense single-stranded RNA virus, belonging to the betacoronavirus genus.¹⁻³ The highly contagious SARS-CoV-2 has spread to more than 190 countries, leading to the COVID-19 pandemic. Currently, COVID-19 poses a major threat to global public health and is regarded as a priority disease by WHO (World Health Organization). Although several vaccines are approved for the emergency use, the new variants pose a great challenge to vaccine efficacy.⁴ The development of antiviral agents against SARS-CoV-2 is urgent. At the beginning of the COVID-19 outbreak, drug repurposing received great attention to find effective antiviral agents for the treatment of COVID-19.⁵ Remdesivir is the first antiviral drug approved by FDA to treat COVID-19, but it had little effect to reduce mortality rate or hospitalization duration for hospitalized patients in the WHO trials⁶. Unfortunately, no other repurposed drugs showed outstanding antiviral activity in clinical trials.^{7, 8}

SARS-CoV-2 is the etiological agent of COVID-19 and its genome encodes 4 structural proteins, 16 non-structural proteins and some accessory proteins.^{9, 10} Among these enzymes, the potential therapeutic targets against SARS-CoV-2 are divided into three groups, including attachment and entry targets, polyproteins processing targets and replicase targets.¹¹ For example, RdRp (RNA-dependent RNA polymerase), M^{pro} (also called 3CL^{pro}), PL^{pro} (Papain-like protease) and nsp14 protein are attractive drug targets to develop small molecule drugs against SARS-CoV-2 because their structures are highly conserved.

The bifunctional nsp14 is essential for viral replication/transcription, which has ExoN (Exonuclease) in the N-terminal domain, possessing the proofreading activity, and N7-MTase (Guanine-N7 methyltransferase) in the C-terminal domain.¹² The N7-MTase domain of nsp14 catalysed the transfer of the methyl group from SAM (S-adenosyl-L-methionine, FIG. 1) to the capped Guanine to form N7-methylguanosine cap and produce the by-product SAH (S-adenosyl-L-homocysteine, FIG. 1).^{13, 14} The viral MTase is an attractive target to develop broad-spectrum antiviral candidates. Potent viral MTase inhibitors were designed based on the modifications in the 5′-methionine or adenine moiety of SAM/SAH. Sinefungin showed submicromolar inhibitory activity against SARS-CoV-2 nsp14 (IC₅₀=0.45 μM)¹⁵, but had no antiviral activity in the cell culture¹⁶. MTTR025495¹⁷ lacking the amino acid moiety exhibited a moderate inhibitory effect with a IC₅₀ of 17 μM. DS0464¹⁷ inhibited nsp14 MTase at the low-micromolar concentration. In addition, derivatives bearing 7-substituted 7-deazaadenine, such as SS148 and 4.1 (FIG. 1) also effectively inhibited nsp14 at the range of submicromolar to nanomolar concentration.^{17, 18} However, the poor cell permeability caused by the zwitterionic moiety prevents their antiviral activity in cell cultures. SAM is a common methyl donor and these MTase inhibitors competitively bind to the SAM/SAH binding site of enzyme, but the development of selective inhibitors is a great challenge because SAM is a common methyl donor of viral and human methyltransferase.

### SUMMARY OF THE INVENTION

The invention relates to 7-substituted 4-aza-7,9-dideazaadenosines. All synthesized compounds were evaluated for activity against SARS-CoV-2 and their cytotoxicity. The 7-vinyl-4-aza-7,9-dideazaadenosine (4.2a) was active against SARS-CoV-2 in Vero 76 cells, displaying an EC₅₀ of 0.16 μg/mL and a selectivity index (SI) of 29.

Interestingly, compound 4.2c bearing the 4-methylstyryl group exhibited similar activity to compound 4.2a, but showed better selectivity (SI₅₀=150).

According to a first aspect of the present invention a compound with general formula (I), or a stereoisomer, tautomer, racemic, salt, hydrate, N-oxide form, or solvate or prodrug thereof

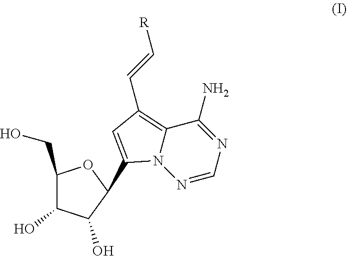

wherein R is selected from the group consisting of hydrogen, C₁₋₆alkyl, C₃₋₁₀cycloalkyl, C₆₋₁₀aryl, C₆₋₁₀arylC₁₋₆alkyl, heterocyclyl and heteroaryl,
wherein said C₁₋₆alkyl, C₃₋₁₀cycloalkyl, C₆₋₁₀aryl, C₆₋₁₀arylC₁₋₆alkyl, heterocyclyl and heteroaryl may be unsubstituted or substituted with halo, C₁₋₆alkyl, haloC₁₋₆alkyl, C₁₋₆alkyloxy, haloC₁₋₆alkyloxy, hydroxyl, amino, monoC₁₋₆alkylamino, diC₁₋₆alkylamino, C₆₋₁₀aryl, carboxy, aminocarbonyl, nitro, cyano.

According to a second aspect, the present invention also encompasses a pharmaceutical composition comprising:
a compound according to the first aspect, or a pharmaceutically acceptable salt, and
at least one pharmaceutical acceptable carrier.

According to a third aspect, the present invention also encompasses a compound according to the first aspect of the invention or a pharmaceutical composition according to the second aspect of the invention, for use as a medicament.

According to a fourth aspect, the present invention also encompasses a compound according to the first aspect of the invention or a pharmaceutical composition according to the second aspect of the present invention, for use in the treatment or prevention of viral infections.

Preferred statements (features) and embodiments of the compounds and processes of this invention are now set forth. Each statements and embodiments of the invention so defined may be combined with any other statement and/or embodiments unless clearly indicated to the contrary. In particular, any feature indicated as being preferred or advantageous may be combined with any other feature or features indicated as being preferred or advantageous.

The invention is further summarized in the following statements:
1. A compound with general formula (I), or a stereoisomer, tautomer, racemic, salt, hydrate, N-oxide form, or solvate or prodrug thereof

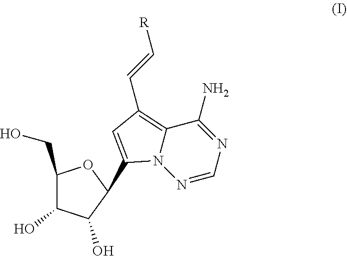

wherein R is selected from the group consisting of hydrogen, C₁₋₆alkyl, C₃₋₁₀cycloalkyl, C₆₋₁₀aryl, C₆₋₁₀arylC₁₋₆alkyl, heterocyclyl and heteroaryl,
wherein said C₁₋₆alkyl, C₃₋₁₀cycloalkyl, C₆₋₁₀aryl, arylC₁₋₆alkyl, heterocyclyl and heteroaryl may be unsubstituted or substituted with halo, C₁₋₆alkyl, haloC₁₋₆alkyl, C₁₋₆alkyloxy, haloC₁₋₆alkyloxy, hydroxyl, amino, monoC₁₋₆alkylamino, diC₁₋₆alkylamino, C₆₋₁₀aryl, carboxy, aminocarbonyl, nitro, cyano.
2. The compound according to statement 1, wherein R is selected from the group consisting of hydrogen, C₁₋₆alkyl, C₃₋₁₀cycloalkyl, C₆₋₁₀aryl, C₆₋₁₀arylC₁₋₆alkyl, heterocyclyl and heteroaryl,
wherein said C₁₋₆alkyl, C₃₋₁₀cycloalkyl, C₆₋₁₀aryl, C₆₋₁₀arylC₁₋₆alkyl, heterocyclyl and heteroaryl may be unsubstituted or substituted with halo, C₁₋₄alkyl, haloC₁₋₄alkyl, C₁₋₄alkyloxy, haloC₁₋₄alkyloxy, hydroxyl, amino, monoC₁₋₄alkylamino, diC₁₋₄alkylamino, C₆₋₁₀aryl, carboxy, aminocarbonyl, nitro, cyano.
3. The compound according to any one of statements 1 or 2, wherein R is selected from the group comprising C₁₋₄alkyl, C₃₋₈cycloalkyl, C₆₋₁₀aryl, C₆₋₁₀arylC₁₋₄alkyl, heterocyclyl and heteroaryl.
4. The compound according to any one of statements 1 or 2, wherein R is selected from the group comprising C₁₋₄alkyl, C₃₋₈cycloalkyl, C₆₋₁₀aryl, C₆₋₁₀arylC₁₋₄alkyl, heterocyclyl and heteroaryl,
wherein said C₁₋₄alkyl, C₃₋₈cycloalkyl, C₆₋₁₀aryl, C₆₋₁₀arylC₁₋₄alkyl, heterocyclyl and heteroaryl may be unsubstituted or substituted with halo, C₁₋₄alkyl, haloC₁₋₄alkyl, C₁₋₄alkyloxy, haloC₁₋₄alkyloxy, hydroxyl, amino, monoC₁₋₄alkylamino, diC₁₋₄alkylamino, C₆₋₁₀aryl, carboxy, aminocarbonyl, nitro, cyano.
5. The compound according to any one of statements 1 to 4, wherein R is selected from the group comprising C₁₋₆alkyl, C₆₋₁₀aryl, and C₆₋₁₀arylC₁₋₆alkyl.
6. The compound according to any one of statements 1 to 5, wherein R is selected from the group comprising C₁₋₄alkyl, C₆₋₁₀aryl, and C₆₋₁₀arylC₁₋₄alkyl.
7. The compound according to any one of statements 1 to 6, wherein R is selected from the group consisting of hydrogen, methyl, ethyl, phenyl, p-methylphenyl, p-ethylphenyl and p-propylphenyl.
8. The compound according to any one of statements 1 to 7, wherein R is hydrogen (4.2a) or p-methylphenyl (4.2c).
9. The compound according to any one of statements 1 to 8, wherein the compound has an E configuration.
10. A pharmaceutical composition comprising:
a compound according to any one of statements 1 to 9, or a pharmaceutically acceptable salt, and
at least one pharmaceutical acceptable carrier.
11. A compound according to any one of statements 1 to 9 or a pharmaceutical composition according to statement 10, for use as a medicament.
12. A compound according to any one of statements 1 to 9 or a pharmaceutical composition according to statement 10, for use in the treatment or prevention of viral infections.
13. The compound for use or pharmaceutical composition for use according to statement 12, wherein the viral infection is an infection by an RNA virus.
14. The compound for use or pharmaceutical composition for use according to statement 13, wherein the RNA virus is selected from the group consisting of coronavirus, measles, tacaribe virus, yellow fever virus, influenzavirus, Chikungunya, dengue, respiratory syncytial virus (RSV), human immunodeficiency virus (HIV) and norovirus.
15. The compound for use or a pharmaceutical composition for use according to statement 14, wherein the coronavirus is MERS or Sars-Cov2.
16. The compound for use or pharmaceutical composition for use according to any one of statements 14 or 15, wherein the coronavirus is SARS-CoV-2.
17. A method of treating and/or preventing a viral infection, comprising the step of administering to an individual a compound with general structure (I) or a pharmaceutically acceptable salt or prodrug thereof according to any one of statements 1 to 9.
18. A process for the preparation of a compound of formula (I) according to any one of statements 1 to 9, comprising the step of
a) halogenation of a compound of formula (A) to produce a compound of formula (B), wherein X is I, Br, Cl or F;

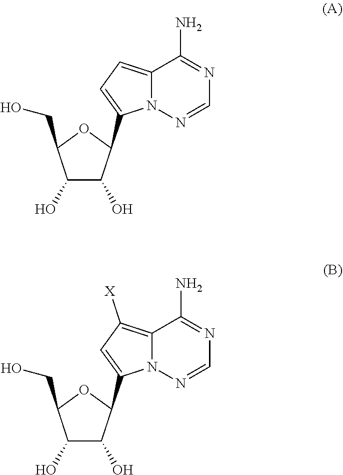

b) contacting a compound of formula (B) with a compound of formula (C) to produce a compound of formula (I); wherein R has the same meaning as in statement 1.

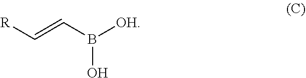

19. The process according to statement 18, wherein step b) is performed in the presence of a palladium catalyst.
20. The process according to statement 19, wherein the palladium catalyst is selected from Pd(PPh₃)₂Cl₂ or Pd(OAc)₂.
21. A compound with general formula I or prodrug thereof, or a stereoisomer, tautomer, racemic, salt, hydrate, N-oxide form, or solvate of said compound or prodrug

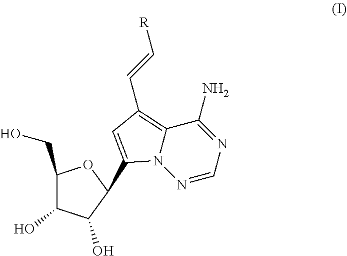

wherein R is selected from the group consisting of hydrogen, methyl, ethyl, phenyl, p-methylphenyl, p-ethylphenyl or p-propylphenyl,
for use in the treatment or prevention of a coronavirus infection.
22. The compound for use according to statement 21, wherein R is hydrogen (4.2a) or p-methylphenyl (4.2c).
23. The compound for use according to statement 24 or 22, wherein the coronavirus is SARS-CoV-2.
24. A compound with general formula I

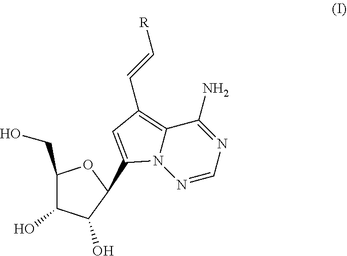

wherein R is selected from the group consisting of hydrogen, methyl, ethyl, phenyl, p-methylphenyl, p-ethylphenyl or p-propylphenyl.
25. The compound according to statement 24, wherein R is hydrogen (4.2a) or p-methylphenyl (4.2c).
26. The compound according to statement 24 or 25, for use as a medicament, more particularly in the treatment or prevention of a viral infection.
27. A compound with general formula I or prodrug thereof, or a stereoisomer, tautomer, racemic, salt, hydrate, N-oxide form, or solvate of said compound or prodrug

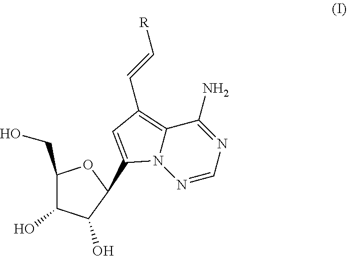

wherein R is selected from the group consisting of hydrogen, methyl, ethyl, benzyl, p-methylbenzyl, p-ethylbenzyl or p-propylbenzyl, for use in the treatment or prevention of a coronavirus infection.
28. The compound for use according to statement 27, wherein R is methyl (4.2a) or p-methylbenzyl (4.2c).
29. A compound with general formula I

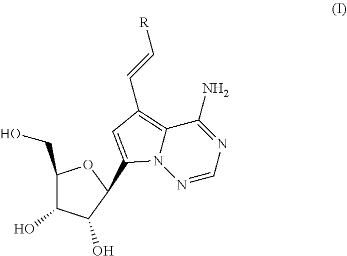

wherein R is selected from the group consisting of hydrogen, methyl, ethyl, benzyl, p-methylbenzyl, p-ethylbenzyl or p-propylbenzyl,
30. The compound according to statement 29, wherein R is methyl (4.2a) or p-methylbenzyl (4.2c).
31. The compound according to statement 29 or 30, for use as a medicament, more particularly in the treatment or prevention of a viral infection.

### DETAILED DESCRIPTION

### FIGURE LEGENDS

FIG. 1. Structure of SAM, SAH and representative methyltransferase (MTase) inhibitors.

FIG. 2. Postulated mechanism of the formation of β-product 4.5.

FIG. 3. Mechanism of Suzuki-Miyaura reaction.

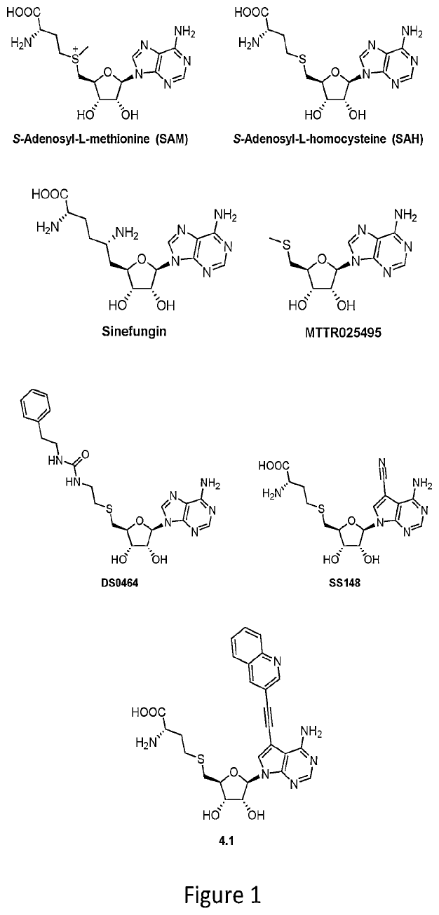

Before the present invention is described, it is to be understood that this invention is not limited to particular processes, methods, and compounds described, as such processes, methods, and compounds may, of course, vary. It is also to be understood that the terminology used herein is not intended to be limiting, since the scope of the present invention will be limited only by the appended claims.

When describing the compounds and processes of the invention, the terms used are to be construed in accordance with the following definitions, unless a context dictates otherwise.

As used in the specification and the appended claims, the singular forms “a”, “an,” and “the” include both singular and plural referents unless the context clearly dictates otherwise. By way of example, “a compound” means one compound or more than one compound.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

As used herein, the term “and/or,” when used in a list of two or more items, means that any one of the listed items can be employed by itself or any combination of two or more of the listed items can be employed. For example, if a list is described as comprising group A, B, and/or C, the list can comprise A alone; B alone; C alone; A and B in combination; A and C in combination, B and C in combination; or A, B, and C in combination.

The recitation of numerical ranges by endpoints includes all integer numbers and, where appropriate, fractions subsumed within that range (e.g. 1 to 5 can include 1, 2, 3, 4 when referring to, for example, a number of elements, and can also include 1.5, 2, 2.75 and 3.80, when referring to, for example, measurements). The recitation of end points also includes the end point values themselves (e.g. from 1.0 to 5.0 includes both 1.0 and 5.0). Any numerical range recited herein is intended to include all sub-ranges subsumed therein.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention.

When describing the present invention, the terms used are to be construed in accordance with the following definitions, unless a context dictates otherwise.

The terms described above and others used in the specification are well understood to those in the art.

The term “pharmaceutically acceptable carrier or excipient” as used herein in relation to pharmaceutical compositions and combined preparations means any material or substance with which the active principle i.e. the compounds of general formula (A), and optionally an antiviral agent and/or an immunosuppressant or immunomodulator may be formulated in order to facilitate its application or dissemination to the locus to be treated, for instance by dissolving, dispersing or diffusing said composition, and/or to facilitate its storage, transport or handling without impairing its effectiveness. The pharmaceutically acceptable carrier may be a solid or a liquid or a gas which has been compressed to form a liquid, i.e. the compositions of this invention can suitably be used as concentrates, emulsions, solutions, granulates, dusts, sprays, aerosols, pellets or powders. Suitable pharmaceutical carriers for use in said pharmaceutical compositions and their formulation are well known to those skilled in the art. There is no particular restriction to their selection within the present invention. Suitable pharmaceutical carriers include additives such as wetting agents, dispersing agents, stickers, adhesives, emulsifying or surface-active agents, thickening agents, complexing agents, gelling agents, solvents, coatings, antibacterial and antifungal agents (for example phenol, sorbic acid, chlorobutanol), isotonic agents (such as sugars or sodium chloride) and the like, provided the same are consistent with pharmaceutical practice, i.e. carriers and additives which do not create permanent damage to mammals.

Whenever the term “substituted” is used herein, it is meant to indicate that one or more hydrogen atoms on the atom indicated in the expression using “substituted” is replaced with a selection from the indicated group, provided that the indicated atom's normal valence is not exceeded, and that the substitution results in a chemically stable compound, i.e. a compound that is sufficiently robust to survive isolation from a reaction mixture. Where groups can be substituted, such groups may be substituted with one or more, and preferably one, two or three substituents.

The terminology “optionally substituted” as used herein is meant to indicate that a group may be unsubstituted or substituted.

The term “halo” or “halogen” as a group or part of a group is generic for fluoro, chloro, bromo, iodo.

The term “amino” refers to the group-NH₂.

The term “hydroxyl” or “hydroxy” as used herein refers to the group —OH.

The term “nitro” as used herein refers to the group —NO₂.

The term “cyano” as used herein refers to the group —CN.

The term “carboxy” or “carboxyl” or “hydroxycarbonyl” as used herein refers to the group —CO₂H.

The term “aminocarbonyl” as used herein refers to the group —CO—NH₂.

The term “alkyl” by itself or as part of another substituent refers to a hydrocarbyl group of formula CₙH₂ₙ₊₁ wherein n is a number greater than or equal to 1. Alkyl groups may be linear or branched and may be substituted as indicated herein. Generally, alkyl groups of this invention comprise from 1 to 6 carbon atoms, preferably from 1 to 5 carbon atoms, preferably from 1 to 4 carbon atoms, more preferably from 1 to 3 carbon atoms, still more preferably 1 to 2 carbon atoms. When a subscript is used herein following a carbon atom, the subscript refers to the number of carbon atoms that the named group may contain. For example, the term “C₁₋₆alkyl”, as a group or part of a group, refers to a hydrocarbyl group of formula —CₙH₂ₙ₊₁ wherein n is a number ranging from 1 to 6. Thus, for example, “C₁₋₆alkyl” includes all linear or branched alkyl groups with between 1 and 6 carbon atoms, and thus includes methyl, ethyl, n-propyl, i-propyl, butyl and its isomers (e.g. n-butyl, i-butyl and t-butyl); pentyl and its isomers, hexyl and its isomers. For example, “C₁₋₅alkyl” includes all includes all linear or branched alkyl groups with between 1 and 5 carbon atoms, and thus includes methyl, ethyl, n-propyl, i-propyl, butyl and its isomers (e.g. n-butyl, i-butyl and t-butyl); pentyl and its isomers. For example, “C₁₋₄alkyl” includes all linear or branched alkyl groups with between 1 and 4 carbon atoms, and thus includes methyl, ethyl, n-propyl, i-propyl, butyl and its isomers (e.g. n-butyl, i-butyl and t-butyl). For example “C₁₋₃alkyl” includes all linear or branched alkyl groups with between 1 and 3 carbon atoms, and thus includes methyl, ethyl, n-propyl, i-propyl. A “substituted C₁₋₆alkyl” refers to a C₁₋₆alkyl group substituted with one or more substituent(s) (for example 1 to 3 substituent(s), for example 1, 2, or 3 substituent(s)) at any available point of attachment.

When the term “alkyl” is used as a suffix following another term, as in “hydroxyalkyl,” this is intended to refer to an alkyl group, as defined above, being substituted with one or two (preferably one) substituent(s) selected from the other, specifically-named group, also as defined herein.

The term “haloC₁₋₆alkyl” as a group or part of a group, refers to a C₁₋₆alkyl group having the meaning as defined above wherein one, two, or three hydrogen atoms are each replaced with a halogen as defined herein. Non-limiting examples of such haloC₁₋₆alkyl groups include chloromethyl, 1-bromoethyl, fluoromethyl, difluoromethyl, trifluoromethyl, 1,1,1-trifluoroethyl and the like.

The term “C₁₋₆alkoxy” or “C₁₋₆alkyloxy”, as a group or part of a group, refers to a group having the formula —OR^{b} wherein R^{b} is C₁₋₆alkyl as defined herein above. Non-limiting examples of suitable C₁₋₆alkoxy include methoxy, ethoxy, propoxy, isopropoxy, butoxy, isobutoxy, sec-butoxy, tert-butoxy, pentyloxy and hexyloxy.

The term “haloC₁₋₆alkoxy”, as a group or part of a group, refers to a group of formula —O—R^{c}, wherein R^{c} is haloC₁₋₆alkyl as defined herein. Non-limiting examples of suitable haloC₁₋₆alkoxy include fluoromethoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethoxy, 2,2,2-trichloroethoxy, trichloromethoxy, 2-bromoethoxy, pentafluoroethyl, 3,3,3-trichloropropoxy, 4,4,4-trichlorobutoxy.

The term “cycloalkyl”, as a group or part of a group, refers to a cyclic alkyl group, that is a monovalent, saturated, hydrocarbyl group having 1 or more cyclic structure, and comprising from 3 to 12 carbon atoms, more preferably from 3 to 9 carbon atoms, more preferably from 3 to 7 carbon atoms; more preferably from 3 to 6 carbon atoms. Cycloalkyl includes all saturated hydrocarbon groups containing 1 or more rings, including monocyclic or bicyclic groups. The further rings of multi-ring cycloalkyls may be either fused, bridged and/or joined through one or more spiro atoms. When a subscript is used herein following a carbon atom, the subscript refers to the number of carbon atoms that the named group may contain. For example, the term “C₃₋₈cycloalkyl”, a cyclic alkyl group comprising from 3 to 8 carbon atoms. For example, the term “C₃₋₆cycloalkyl”, a cyclic alkyl group comprising from 3 to 6 carbon atoms. Examples of C₃₋₁₂cycloalkyl groups include but are not limited to cyclopropyl, cyclobutyl, cyclopentyl, cyclohexyl, cycloheptyl, cyclooctyl, bicycle[2.2.1]heptan-2yl, (1S,4R)-norbornan-2-yl, (1R,4R)-norbornan-2-yl, (1S,4S)-norbornan-2-yl, (1R,4S)-norbornan-2-yl.

The term “C₆₋₁₂aryl”, as a group or part of a group, refers to a polyunsaturated, aromatic hydrocarbyl group having a single ring (i.e. phenyl) or multiple aromatic rings fused together (e.g. naphthyl), or linked covalently, typically containing 6 to 12 atoms; preferably 6 to 10, wherein at least one ring is aromatic. The aromatic ring may optionally include one to two additional rings (either cycloalkyl, heterocyclyl or heteroaryl) fused thereto. Examples of suitable aryl include C₆₋₁₀aryl, more preferably C₆₋₈aryl. Non-limiting examples of C₆₋₁₂aryl comprise phenyl; biphenylyl; biphenylenyl; or 1- or 2-naphthanelyl; 1-, 2-, 3-, 4-, 5- or 6-tetralinyl (also known as “1,2,3,4-tetrahydronaphthalene); 1-, 2-, 3-, 4-, 5-, 6-, 7- or 8-azulenyl, 4-, 5-, 6 or 7-indenyl, 4- or 5-indanyl, 5-, 6-, 7- or 8-tetrahydronaphthyl; 1,2,3,4-tetrahydronaphthyl; and 1,4-dihydronaphthyl; 1-, 2-, 3-, 4- or 5-pyrenyl. A “substituted C₆₋₁₂aryl” refers to a C₆₋₁₂aryl group having one or more substituent(s) (for example 1, 2 or 3 substituent(s), or 1 to 2 substituent(s)), at any available point of attachment.

The term “C₆₋₁₂arylC₁₋₆alkyl”, as a group or part of a group, means a C₁₋₆alkyl as defined herein, wherein at least one hydrogen atom is replaced by at least one C₆₋₁₂aryl as defined herein. Non-limiting examples of C₆₋₁₂arylC₁₋₆alkyl group include benzyl, phenethyl, dibenzylmethyl, methylphenylmethyl, 3-(2-naphthyl)-butyl, and the like.

The terms “heterocyclyl” or “heterocycloakyl” or “heterocyclo”, as a group or part of a group, refer to non-aromatic, fully saturated or partially unsaturated cyclic groups (for example, 3 to 7 member monocyclic, 7 to 11 member bicyclic, or comprising a total of 3 to 10 ring atoms) which have at least one heteroatom in at least one carbon atom-containing ring; wherein said ring may be fused to an aryl, cycloalkyl, heteroaryl or heterocyclyl ring. Each ring of the heterocyclyl group containing a heteroatom may have 1, 2, 3 or 4 heteroatoms selected from N, O and/or S, where the N and S heteroatoms may optionally be oxidized and the N heteroatoms may optionally be quaternized; and wherein at least one carbon atom of heterocyclyl can be oxidized to form at least one C═O. The heterocyclic group may be attached at any heteroatom or carbon atom of the ring or ring system, where valence allows. The rings of multi-ring heterocycles may be fused, bridged and/or joined through one or more spiro atoms.

Non limiting exemplary heterocyclic groups include aziridinyl, oxiranyl, thiiranyl, piperidinyl, azetidinyl, oxetanyl, pyrrolidinyl, thietanyl, 2-imidazolinyl, pyrazolidinyl imidazolidinyl, isoxazolinyl, oxazolidinyl, isoxazolidinyl, thiazolidinyl, isothiazolidinyl, piperidinyl, succinimidyl, 3H-indolyl, indolinyl, isoindolinyl, chromanyl (also known as 3,4-dihydrobenzo[b]pyranyl), 2H-pyrrolyl, 1-pyrrolinyl, 2-pyrrolinyl, 3-pyrrolinyl, 4H-quinolizinyl, 2-oxopiperazinyl, piperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, 3-dioxolanyl, 1,4-dioxanyl, 2,5-dioximidazolidinyl, 2-oxopiperidinyl, 2-oxopyrrolodinyl, indolinyl, tetrahydropyranyl, tetrahydrofuranyl, tetrahydrothiophenyl, tetrahydroquinolinyl, tetrahydroisoquinolin-1-yl, tetrahydroisoquinolin-2-yl, tetrahydroisoquinolin-3-yl, tetrahydroisoquinolin-4-yl, thiomorpholin-4-yl, thiomorpholin-4-ylsulfoxide, thiomorpholin-4-ylsulfone, 1,3-dioxolanyl, 1,4-oxathianyl, 1,4-dithianyl, 1,3,5-trioxanyl, 1H-pyrrolizinyl, tetrahydro-1,1-dioxothiophenyl, N-formylpiperazinyl, and morpholin-4-yl. The term “aziridinyl” as used herein includes aziridin-1-yl and aziridin-2-yl. The term “oxyranyl” as used herein includes oxyranyl-2-yl. The term “thiiranyl” as used herein includes thiiran-2-yl. The term “azetidinyl” as used herein includes azetidin-1-yl, azetidin-2-yl and azetidin-3-yl. The term “oxetanyl” as used herein includes oxetan-2-yl and oxetan-3-yl. The term “thietanyl” as used herein includes thietan-2-yl and thietan-3-yl. The term “pyrrolidinyl” as used herein includes pyrrolidin-1-yl, pyrrolidin-2-yl and pyrrolidin-3-yl. The term “tetrahydrofuranyl” is used herein includes tetrahydrofuran-2-yl and tetrahydrofuran-3-yl. The term “tetrahydrothiophenyl” as used herein includes tetrahydrothiophen-2-yl and tetrahydrothiophen-3-yl. The term “succinimidyl” as used herein includes succinimid-1-yl and succininmid-3-yl. The term “dihydropyrrolyl” as used herein includes 2,3-dihydropyrrol-1-yl, 2,3-dihydro-1H-pyrrol-2-yl, 2,3-dihydro-1H-pyrrol-3-yl, 2,5-dihydropyrrol-1-yl, 2,5-dihydro-1H-pyrrol-3-yl and 2,5-dihydropyrrol-5-yl. The term “2H-pyrrolyl” as used herein includes 2H-pyrrol-2-yl, 2H-pyrrol-3-yl, 2H-pyrrol-4-yl and 2H-pyrrol-5-yl. The term “3H-pyrrolyl” as used herein includes 3H-pyrrol-2-yl, 3H-pyrrol-3-yl, 3H-pyrrol-4-yl and 3H-pyrrol-5-yl. The term “dihydrofuranyl” as used herein includes 2,3-dihydrofuran-2-yl, 2,3-dihydrofuran-3-yl, 2,3-dihydrofuran-4-yl, 2,3-dihydrofuran-5-yl, 2,5-dihydrofuran-2-yl, 2,5-dihydrofuran-3-yl, 2,5-dihydrofuran-4-yl and 2,5-dihydrofuran-5-yl. The term “dihydrothiophenyl” as used herein includes 2,3-dihydrothiophen-2-yl, 2,3-dihydrothiophen-3-yl, 2,3-dihydrothiophen-4-yl, 2,3-dihydrothiophen-5-yl, 2,5-dihydrothiophen-2-yl, 2,5-dihydrothiophen-3-yl, 2,5-dihydrothiophen-4-yl and 2,5-dihydrothiophen-5-yl. The term “imidazolidinyl” as used herein includes imidazolidin-1-yl, imidazolidin-2-yl and imidazolidin-4-yl. The term “pyrazolidinyl” as used herein includes pyrazolidin-1-yl, pyrazolidin-3-yl and pyrazolidin-4-yl. The term “imidazolinyl” as used herein includes imidazolin-1-yl, imidazolin-2-yl, imidazolin-4-yl and imidazolin-5-yl. The term “pyrazolinyl” as used herein includes 1-pyrazolin-3-yl, 1-pyrazolin-4-yl, 2-pyrazolin-1-yl, 2-pyrazolin-3-yl, 2-pyrazolin-4-yl, 2-pyrazolin-5-yl, 3-pyrazolin-1-yl, 3-pyrazolin-2-yl, 3-pyrazolin-3-yl, 3-pyrazolin-4-yl and 3-pyrazolin-5-yl. The term “dioxolanyl” also known as “1,3-dioxolanyl” as used herein includes dioxolan-2-yl, dioxolan-4-yl and dioxolan-5-yl.

The term “dioxolyl” also known as “1,3-dioxolyl” as used herein includes dioxol-2-yl, dioxol-4-yl and dioxol-5-yl. The term “oxazolidinyl” as used herein includes oxazolidin-2-yl, oxazolidin-3-yl, oxazolidin-4-yl and oxazolidin-5-yl. The term “isoxazolidinyl” as used herein includes isoxazolidin-2-yl, isoxazolidin-3-yl, isoxazolidin-4-yl and isoxazolidin-5-yl. The term “oxazolinyl” as used herein includes 2-oxazolinyl-2-yl, 2-oxazolinyl-4-yl, 2-oxazolinyl-5-yl, 3-oxazolinyl-2-yl, 3-oxazolinyl-4-yl, 3-oxazolinyl-5-yl, 4-oxazolinyl-2-yl, 4-oxazolinyl-3-yl, 4-oxazolinyl-4-yl and 4-oxazolinyl-5-yl. The term “isoxazolinyl” as used herein includes 2-isoxazolinyl-3-yl, 2-isoxazolinyl-4-yl, 2-isoxazolinyl-5-yl, 3-isoxazolinyl-3-yl, 3-isoxazolinyl-4-yl, 3-isoxazolinyl-5-yl, 4-isoxazolinyl-2-yl, 4-isoxazolinyl-3-yl, 4-isoxazolinyl-4-yl and 4-isoxazolinyl-5-yl. The term “thiazolidinyl” as used herein includes thiazolidin-2-yl, thiazolidin-3-yl, thiazolidin-4-yl and thiazolidin-5-yl. The term “isothiazolidinyl” as used herein includes isothiazolidin-2-yl, isothiazolidin-3-yl, isothiazolidin-4-yl and isothiazolidin-5-yl. The term “thiazolinyl” as used herein includes 2-thiazolinyl-2-yl, 2-thiazolinyl-4-yl, 2-thiazolinyl-5-yl, 3-thiazolinyl-2-yl, 3-thiazolinyl-4-yl, 3-thiazolinyl-5-yl, 4-thiazolinyl-2-yl, 4-thiazolinyl-3-yl, 4-thiazolinyl-4-yl and 4-thiazolinyl-5-yl. The term “isothiazolinyl” as used herein includes 2-isothiazolinyl-3-yl, 2-isothiazolinyl-4-yl, 2-isothiazolinyl-5-yl, 3-isothiazolinyl-3-yl, 3-isothiazolinyl-4-yl, 3-isothiazolinyl-5-yl, 4-isothiazolinyl-2-yl, 4-isothiazolinyl-3-yl, 4-isothiazolinyl-4-yl and 4-isothiazolinyl-5-yl. The term “piperidyl” also known as “piperidinyl” as used herein includes piperid-1-yl, piperid-2-yl, piperid-3-yl and piperid-4-yl. The term “dihydropyridinyl” as used herein includes 1,2-dihydropyridin-1-yl, 1,2-dihydropyridin-2-yl, 1,2-dihydropyridin-3-yl, 1,2-dihydropyridin-4-yl, 1,2-dihydropyridin-5-yl, 1,2-dihydropyridin-6-yl, 1,4-dihydropyridin-1-yl, 1,4-dihydropyridin-2-yl, 1,4-dihydropyridin-3-yl, 1,4-dihydropyridin-4-yl, 2,3-dihydropyridin-2-yl, 2,3-dihydropyridin-3-yl, 2,3-dihydropyridin-4-yl, 2,3-dihydropyridin-5-yl, 2,3-dihydropyridin-6-yl, 2,5-dihydropyridin-2-yl, 2,5-dihydropyridin-3-yl, 2,5-dihydropyridin-4-yl, 2,5-dihydropyridin-5-yl, 2,5-dihydropyridin-6-yl, 3,4-dihydropyridin-2-yl, 3,4-dihydropyridin-3-yl, 3,4-dihydropyridin-4-yl, 3,4-dihydropyridin-5-yl and 3,4-dihydropyridin-6-yl. The term “tetrahydropyridinyl” as used herein includes 1,2,3,4-tetrahydropyridin-1-yl, 1,2,3,4-tetrahydropyridin-2-yl, 1,2,3,4-tetrahydropyridin-3-yl, 1,2,3,4-tetrahydropyridin-4-yl, 1,2,3,4-tetrahydropyridin-5-yl, 1,2,3,4-tetrahydropyridin-6-yl, 1,2,3,6-tetrahydropyridin-1-yl, 1,2,3,6-tetrahydropyridin-2-yl, 1,2,3,6-tetrahydropyridin-3-yl, 1,2,3,6-tetrahydropyridin-4-yl, 1,2,3,6-tetrahydropyridin-5-yl, 1,2,3,6-tetrahydropyridin-6-yl, 2,3,4,5-tetrahydropyridin-2-yl, 2,3,4,5-tetrahydropyridin-3-yl, 2,3,4,5-tetrahydropyridin-3-yl, 2,3,4,5-tetrahydropyridin-4-yl, 2,3,4,5-tetrahydropyridin-5-yl and 2,3,4,5-tetrahydropyridin-6-yl. The term “tetrahydropyranyl” also known as “oxanyl” or “tetrahydro-2H-pyranyl”, as used herein includes tetrahydropyran-2-yl, tetrahydropyran-3-yl and tetrahydropyran-4-yl. The term “2H-pyranyl” as used herein includes 2H-pyran-2-yl, 2H-pyran-3-yl, 2H-pyran-4-yl, 2H-pyran-5-yl and 2H-pyran-6-yl. The term “4H-pyranyl” as used herein includes 4H-pyran-2-yl, 4H-pyran-3-yl and 4H-pyran-4-yl. The term “3,4-dihydro-2H-pyranyl” as used herein includes 3,4-dihydro-2H-pyran-2-yl, 3,4-dihydro-2H-pyran-3-yl, 3,4-dihydro-2H-pyran-4-yl, 3,4-dihydro-2H-pyran-5-yl and 3,4-dihydro-2H-pyran-6-yl. The term “3,6-dihydro-2H-pyranyl” as used herein includes 3,6-dihydro-2H-pyran-2-yl, 3,6-dihydro-2H-pyran-3-yl, 3,6-dihydro-2H-pyran-4-yl, 3,6-dihydro-2H-pyran-5-yl and 3,6-dihydro-2H-pyran-6-yl. The term “tetrahydrothiophenyl”, as used herein includes tetrahydrothiophen-2-yl, tetrahydrothiophenyl-3-yl and tetrahydrothiophenyl-4-yl. The term “2H-thiopyranyl” as used herein includes 2H-thiopyran-2-yl, 2H-thiopyran-3-yl, 2H-thiopyran-4-yl, 2H-thiopyran-5-yl and 2H-thiopyran-6-yl. The term “4H-thiopyranyl” as used herein includes 4H-thiopyran-2-yl, 4H-thiopyran-3-yl and 4H-thiopyran-4-yl. The term “3,4-dihydro-2H-thiopyranyl” as used herein includes 3,4-dihydro-2H-thiopyran-2-yl, 3,4-dihydro-2H-thiopyran-3-yl, 3,4-dihydro-2H-thiopyran-4-yl, 3,4-dihydro-2H-thiopyran-5-yl and 3,4-dihydro-2H-thiopyran-6-yl. The term “3,6-dihydro-2H-thiopyranyl” as used herein includes 3,6-dihydro-2H-thiopyran-2-yl, 3,6-dihydro-2H-thiopyran-3-yl, 3,6-dihydro-2H-thiopyran-4-yl, 3,6-dihydro-2H-thiopyran-5-yl and 3,6-dihydro-2H-thiopyran-6-yl. The term “piperazinyl” also known as “piperazidinyl” as used herein includes piperazin-1-yl and piperazin-2-yl. The term “morpholinyl” as used herein includes morpholin-2-yl, morpholin-3-yl and morpholin-4-yl. The term “thiomorpholinyl” as used herein includes thiomorpholin-2-yl, thiomorpholin-3-yl and thiomorpholin-4-yl. The term “dioxanyl” as used herein includes 1,2-dioxan-3-yl, 1,2-dioxan-4-yl, 1,3-dioxan-2-yl, 1,3-dioxan-4-yl, 1,3-dioxan-5-yl and 1,4-dioxan-2-yl. The term “dithianyl” as used herein includes 1,2-dithian-3-yl, 1,2-dithian-4-yl, 1,3-dithian-2-yl, 1,3-dithian-4-yl, 1,3-dithian-5-yl and 1,4-dithian-2-yl. The term “oxathianyl” as used herein includes oxathian-2-yl and oxathian-3-yl. The term “trioxanyl” as used herein includes 1,2,3-trioxan-4-yl, 1,2,3-trioxay-5-yl, 1,2,4-trioxay-3-yl, 1,2,4-trioxay-5-yl, 1,2,4-trioxay-6-yl and 1,3,4-trioxay-2-yl. The term “azepanyl” as used herein includes azepan-1-yl, azepan-2-yl, azepan-1-yl, azepan-3-yl and azepan-4-yl. The term “homopiperazinyl” as used herein includes homopiperazin-1-yl, homopiperazin-2-yl, homopiperazin-3-yl and homopiperazin-4-yl. The term “indolinyl” as used herein includes indolin-1-yl, indolin-2-yl, indolin-3-yl, indolin-4-yl, indolin-5-yl, indolin-6-yl, and indolin-7-yl. The term “quinolizinyl” as used herein includes quinolizidin-1-yl, quinolizidin-2-yl, quinolizidin-3-yl and quinolizidin-4-yl. The term “isoindolinyl” as used herein includes isoindolin-1-yl, isoindolin-2-yl, isoindolin-3-yl, isoindolin-4-yl, isoindolin-5-yl, isoindolin-6-yl, and isoindolin-7-yl. The term “3H-indolyl” as used herein includes 3H-indol-2-yl, 3H-indol-3-yl, 3H-indol-4-yl, 3H-indol-5-yl, 3H-indol-6-yl, and 3H-indol-7-yl. The term “quinolizinyl” as used herein includes quinolizidin-1-yl, quinolizidin-2-yl, quinolizidin-3-yl and quinolizidin-4-yl. The term “quinolizinyl” as used herein includes quinolizidin-1-yl, quinolizidin-2-yl, quinolizidin-3-yl and quinolizidin-4-yl. The term “tetrahydroquinolinyl” as used herein includes tetrahydroquinolin-1-yl, tetrahydroquinolin-2-yl, tetrahydroquinolin-3-yl, tetrahydroquinolin-4-yl, tetrahydroquinolin-5-yl, tetrahydroquinolin-6-yl, tetrahydroquinolin-7-yl and tetrahydroquinolin-8-yl. The term “tetrahydroisoquinolinyl” as used herein includes tetrahydroisoquinolin-1-yl, tetrahydroisoquinolin-2-yl, tetrahydroisoquinolin-3-yl, tetrahydroisoquinolin-4-yl, tetrahydroisoquinolin-5-yl, tetrahydroisoquinolin-6-yl, tetrahydroisoquinolin-7-yl and tetrahydroisoquinolin-8-yl. The term “chromanyl” as used herein includes chroman-2-yl, chroman-3-yl, chroman-4-yl, chroman-5-yl, chroman-6-yl, chroman-7-yl and chroman-8-yl. The term “1H-pyrrolizine” as used herein includes 1H-pyrrolizin-1-yl, 1H-pyrrolizin-2-yl, 1H-pyrrolizin-3-yl, 1H-pyrrolizin-5-yl, 1H-pyrrolizin-6-yl and 1H-pyrrolizin-7-yl. The term “3H-pyrrolizine” as used herein includes 3H-pyrrolizin-1-yl, 3H-pyrrolizin-2-yl, 3H-pyrrolizin-3-yl, 3H-pyrrolizin-5-yl, 3H-pyrrolizin-6-yl and 3H-pyrrolizin-7-yl.

The term “heteroaryl” as a group or part of a group, refers but is not limited to 5 to 12 carbon-atom aromatic rings or ring systems containing 1 or 2 rings which can be fused together or linked covalently, typically containing 5 to 6 atoms; at least one of which is aromatic in which one or more carbon atoms in one or more of these rings can be replaced by N, O and/or S atoms where the N and S heteroatoms may optionally be oxidized and the N heteroatoms may optionally be quaternized, and wherein at least one carbon atom of said heteroaryl can be oxidized to form at least one C═O. Such rings may be fused to an aryl, cycloalkyl, heteroaryl or heterocyclyl ring. Non-limiting examples of such heteroaryl, include: pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, benzo[d]oxazol-2(3H)-one, 2,3-dihydro-benzofuranyl, thienopyridinyl, purinyl, imidazo[1,2-a]pyridinyl, 6-oxo-pyridazin-1(6H)-yl, 2-oxopyridin-1(2H)-yl, 6-oxo-pyridazin-1(6H)-yl, 2-oxopyridin-1(2H)-yl, 1,3-benzodioxolyl, quinolinyl, isoquinolinyl, cinnolinyl, quinazolinyl, quinoxalinyl; preferably said heteroaryl group is selected from the group consisting of pyridyl, 1,3-benzodioxolyl, benzo[d]oxazol-2(3H)-one, 2,3-dihydro-benzofuranyl, pyrazinyl, pyrazolyl, pyrrolyl, isoxazolyl, thiophenyl, imidazolyl, benzimidazolyl, pyrimidinyl, triazolyl and thiazolyl.

The term “pyrrolyl” (also called azolyl) as used herein includes pyrrol-1-yl, pyrrol-2-yl and pyrrol-3-yl. The term “furanyl” (also called “furyl”) as used herein includes furan-2-yl and furan-3-yl (also called furan-2-yl and furan-3-yl). The term “thiophenyl” (also called “thienyl”) as used herein includes thiophen-2-yl and thiophen-3-yl (also called thien-2-yl and thien-3-yl). The term “pyrazolyl” (also called 1H-pyrazolyl and 1,2-diazolyl) as used herein includes pyrazol-1-yl, pyrazol-3-yl, pyrazol-4-yl and pyrazol-5-yl. The term “imidazolyl” as used herein includes imidazol-1-yl, imidazol-2-yl, imidazol-4-yl and imidazol-5-yl. The term “oxazolyl” (also called 1,3-oxazolyl) as used herein includes oxazol-2-yl, oxazol-4-yl and oxazol-5-yl. The term “isoxazolyl” (also called 1,2-oxazolyl), as used herein includes isoxazol-3-yl, isoxazol-4-yl, and isoxazol-5-yl. The term “thiazolyl” (also called 1,3-thiazolyl), as used herein includes thiazol-2-yl, thiazol-4-yl and thiazol-5-yl (also called 2-thiazolyl, 4-thiazolyl and 5-thiazolyl). The term “isothiazolyl” (also called 1,2-thiazolyl) as used herein includes isothiazol-3-yl, isothiazol-4-yl, and isothiazol-5-yl. The term “triazolyl” as used herein includes 1H-triazolyl and 4H-1,2,4-triazolyl, “1H-triazolyl” includes 1H-1,2,3-triazol-1-yl, 1H-1,2,3-triazol-4-yl, 1H-1,2,3-triazol-5-yl, 1H-1,2,4-triazol-1-yl, 1H-1,2,4-triazol-3-yl and 1H-1,2,4-triazol-5-yl. “4H-1,2,4-triazolyl” includes 4H-1,2,4-triazol-4-yl, and 4H-1,2,4-triazol-3-yl. The term “oxadiazolyl” as used herein includes 1,2,3-oxadiazol-4-yl, 1,2,3-oxadiazol-5-yl, 1,2,4-oxadiazol-3-yl, 1,2,4-oxadiazol-5-yl, 1,2,5-oxadiazol-3-yl and 1,3,4-oxadiazol-2-yl. The term “thiadiazolyl” as used herein includes 1,2,3-thiadiazol-4-yl, 1,2,3-thiadiazol-5-yl, 1,2,4-thiadiazol-3-yl, 1,2,4-thiadiazol-5-yl, 1,2,5-thiadiazol-3-yl (also called furazan-3-yl) and 1,3,4-thiadiazol-2-yl. The term “tetrazolyl” as used herein includes 1H-tetrazol-1-yl, 1H-tetrazol-5-yl, 2H-tetrazol-2-yl, and 2H-tetrazol-5-yl. The term “oxatriazolyl” as used herein includes 1,2,3,4-oxatriazol-5-yl and 1,2,3,5-oxatriazol-4-yl. The term “thiatriazolyl” as used herein includes 1,2,3,4-thiatriazol-5-yl and 1,2,3,5-thiatriazol-4-yl. The term “pyridinyl” (also called “pyridyl”) as used herein includes pyridin-2-yl, pyridin-3-yl and pyridin-4-yl (also called 2-pyridyl, 3-pyridyl and 4-pyridyl). The term “pyrimidyl” as used herein includes pyrimid-2-yl, pyrimid-4-yl, pyrimid-5-yl and pyrimid-6-yl. The term “pyrazinyl” as used herein includes pyrazin-2-yl and pyrazin-3-yl. The term “pyridazinyl as used herein includes pyridazin-3-yl and pyridazin-4-yl. The term “oxazinyl” (also called “1,4-oxazinyl”) as used herein includes 1,4-oxazin-4-yl and 1,4-oxazin-5-yl. The term “dioxinyl” (also called “1,4-dioxinyl”) as used herein includes 1,4-dioxin-2-yl and 1,4-dioxin-3-yl. The term “thiazinyl” (also called “1,4-thiazinyl”) as used herein includes 1,4-thiazin-2-yl, 1,4-thiazin-3-yl, 1,4-thiazin-4-yl, 1,4-thiazin-5-yl and 1,4-thiazin-6-yl. The term “triazinyl” as used herein includes 1,3,5-triazin-2-yl, 1,2,4-triazin-3-yl, 1,2,4-triazin-5-yl, 1,2,4-triazin-6-yl, 1,2,3-triazin-4-yl and 1,2,3-triazin-5-yl. The term “imidazo[2,1-b][1,3]thiazolyl” as used herein includes imidazo[2,1-b][1,3]thiazoi-2-yl, imidazo[2,1-b][1,3]thiazol-3-yl, imidazo[2,1-b][1,3]thiazol-5-yl and imidazo [2,1-b][1,3]thiazol-6-yl. The term “thieno[3,2-b]furanyl” as used herein includes thieno[3,2-b]furan-2-yl, thieno[3,2-b]furan-3-yl, thieno[3,2-b]furan-4-yl, and thieno[3,2-b]furan-5-yl. The term “thieno[3,2-b]thiophenyl” as used herein includes thieno[3,2-b]thien-2-yl, thieno[3,2-b]thien-3-yl, thieno[3,2-b]thien-5-yl and thieno[3,2-b]thien-6-yl. The term “thieno[2,3-d][1,3]thiazolyl” as used herein includes thieno[2,3-d][1,3]thiazol-2-yl, thieno[2,3-d][1,3]thiazol-5-yl and thieno[2,3-d][1,3]thiazol-6-yl. The term “thieno[2,3-d]imidazolyl” as used herein includes thieno[2,3-d]imidazol-2-yl, thieno[2,3-d] imidazol-4-yl and thieno[2,3-d]imidazol-5-yl. The term “tetrazolo[1,5-a]pyridinyl” as used herein includes tetrazolo[1,5-a]pyridine-5-yl, tetrazolo[1,5-a]pyridine-6-yl, tetrazolo[1,5-a]pyridine-7-yl, and tetrazolo[1,5-a]pyridine-8-yl. The term “indolyl” as used herein includes indol-1-yl, indol-2-yl, indol-3-yl,-indol-4-yl, indol-5-yl, indol-6-yl and indol-7-yl. The term “indolizinyl” as used herein includes indolizin-1-yl, indolizin-2-yl, indolizin-3-yl, indolizin-5-yl, indolizin-6-yl, indolizin-7-yl, and indolizin-8-yl. The term “isoindolyl” as used herein includes isoindol-1-yl, isoindol-2-yl, isoindol-3-yl, isoindol-4-yl, isoindol-5-yl, isoindol-6-yl and isoindol-7-yl. The term “benzofuranyl” (also called benzo[b]furanyl) as used herein includes benzofuran-2-yl, benzofuran-3-yl, benzofuran-4-yl, benzofuran-5-yl, benzofuran-6-yl and benzofuran-7-yl. The term “isobenzofuranyl” (also called benzo[c]furanyl) as used herein includes isobenzofuran-1-yl, isobenzofuran-3-yl, isobenzofuran-4-yl, isobenzofuran-5-yl, isobenzofuran-6-yl and isobenzofuran-7-yl. The term “benzothiophenyl” (also called benzo[b]thienyl) as used herein includes 2-benzo[b]thiophenyl, 3-benzo[b]thiophenyl, 4-benzo[b]thiophenyl, 5-benzo[b]thiophenyl, 6-benzo[b]thiophenyl and-7-benzo[b]thiophenyl (also called benzothien-2-yl, benzothien-3-yl, benzothien-4-yl, benzothien-5-yl, benzothien-6-yl and benzothien-7-yl). The term “isobenzothiophenyl” (also called benzo[c]thienyl) as used herein includes isobenzothien-1-yl, isobenzothien-3-yl, isobenzothien-4-yl, isobenzothien-5-yl, isobenzothien-6-yl and isobenzothien-7-yl. The term “indazolyl” (also called 1H-indazolyl or 2-azaindolyl) as used herein includes 1H-indazol-1-yl, 1H-indazol-3-yl, 1H-indazol-4-yl, 1H-indazol-5-yl, 1H-indazol-6-yl, 1H-indazol-7-yl, 2H-indazol-2-yl, 2H-indazol-3-yl, 2H-indazol-4-yl, 2H-indazol-5-yl, 2H-indazol-6-yl, and 2H-indazol-7-yl. The term “benzimidazolyl” as used herein includes benzimidazol-1-yl, benzimidazol-2-yl, benzimidazol-4-yl, benzimidazol-5-yl, benzimidazol-6-yl and benzimidazol-7-yl. The term “1,3-benzoxazolyl” as used herein includes 1,3-benzoxazol-2-yl, 1,3-benzoxazol-4-yl, 1,3-benzoxazol-5-yl, 1,3-benzoxazol-6-yl and 1,3-benzoxazol-7-yl. The term “1,2-benzisoxazolyl” as used herein includes 1,2-benzisoxazol-3-yl, 1,2-benzisoxazol-4-yl, 1,2-benzisoxazol-5-yl, 1,2-benzisoxazol-6-yl and 1,2-benzisoxazol-7-yl. The term “2,1-benzisoxazolyl” as used herein includes 2,1-benzisoxazol-3-yl, 2,1-benzisoxazol-4-yl, 2,1-benzisoxazol-5-yl, 2,1-benzisoxazol-6-yl and 2,1-benzisoxazol-7-yl. The term “1,3-benzothiazolyl” as used herein includes 1,3-benzothiazol-2-yl, 1,3-benzothiazol-4-yl, 1,3-benzothiazol-5-yl, 1,3-benzothiazol-6-yl and 1,3-benzothiazol-7-yl. The term “1,2-benzoisothiazolyl” as used herein includes 1,2-benzisothiazol-3-yl, 1,2-benzisothiazol-4-yl, 1,2-benzisothiazol-5-yl, 1,2-benzisothiazol-6-yl and 1,2-benzisothiazol-7-yl. The term “2,1-benzoisothiazolyl” as used herein includes 2,1-benzisothiazol-3-yl, 2,1-benzisothiazol-4-yl, 2,1-benzisothiazol-5-yl, 2,1-benzisothiazol-6-yl and 2,1-benzisothiazol-7-yl. The term “benzotriazolyl” as used herein includes benzotriazol-1-yl, benzotriazol-4-yl, benzotriazol-5-yl, benzotriazol-6-yl and benzotriazol-7-yl. The term “1,2,3-benzoxadiazolyl” as used herein includes 1,2,3-benzoxadiazol-4-yl, 1,2,3-benzoxadiazol-5-yl, 1,2,3-benzoxadiazol-6-yl and 1,2,3-benzoxadiazol-7-yl. The term “2,1,3-benzoxadiazolyl” as used herein includes 2,1,3-benzoxadiazol-4-yl, 2,1,3-benzoxadiazol-5-yl, 2,1,3-benzoxadiazol-6-yl and 2,1,3-benzoxadiazol-7-yl. The term “1,2,3-benzothiadiazolyl” as used herein includes 1,2,3-benzothiadiazol-4-yl, 1,2,3-benzothiadiazol-5-yl, 1,2,3-benzothiadiazol-6-yl and 1,2,3-benzothiadiazol-7-yl. The term “2,1,3-benzothiadiazolyl” as used herein includes 2,1,3-benzothiadiazol-4-yl, 2,1,3-benzothiadiazol-5-yl, 2,1,3-benzothiadiazol-6-yl and 2,1,3-benzothiadiazol-7-yl. The term “thienopyridinyl” as used herein includes thieno[2,3-b]pyridinyl, thieno[2,3-c]pyridinyl, thieno[3,2-c]pyridinyl and thieno[3,2-b]pyridinyl. The term “purinyl” as used herein includes purin-2-yl, purin-6-yl, purin-7-yl and purin-8-yl. The term “imidazo[1,2-a]pyridinyl”, as used herein includes imidazo[1,2-a]pyridin-2-yl, imidazo[1,2-a]pyridin-3-yl, imidazo[1,2-a]pyridin-4-yl, imidazo[1,2-a]pyridin-5-yl, imidazo[1,2-a]pyridin-6-yl and imidazo[1,2-a]pyridin-7-yl. The term “1,3-benzodioxolyl”, as used herein includes 1,3-benzodioxol-4-yl, 1,3-benzodioxol-5-yl, 1,3-benzodioxol-6-yl, and 1,3-benzodioxol-7-yl. The term “quinolinyl” as used herein includes quinolin-2-yl, quinolin-3-yl, quinolin-4-yl, quinolin-5-yl, quinolin-6-yl, quinolin-7-yl and quinolin-8-yl. The term “isoquinolinyl” as used herein includes isoquinolin-1-yl, isoquinolin-3-yl, isoquinolin-4-yl, isoquinolin-5-yl, isoquinolin-6-yl, isoquinolin-7-yl and isoquinolin-8-yl. The term “cinnolinyl” as used herein includes cinnolin-3-yl, cinnolin-4-yl, cinnolin-5-yl, cinnolin-6-yl, cinnolin-7-yl and cinnolin-8-yl. The term “quinazolinyl” as used herein includes quinazolin-2-yl, quinazolin-4-yl, quinazolin-5-yl, quinazolin-6-yl, quinazolin-7-yl and quinazolin-8-yl. The term “quinoxalinyl” as used herein includes quinoxalin-2-yl, quinoxalin-5-yl, and quinoxalin-6-yl.

The term “mono- or di-C₁₋₆alkylamino”, as a group or part of a group, refers to a group of formula —N(R^{o})(R^{p}) wherein R^{o} and R^{p} are each independently selected from hydrogen, or C₁₋₆alkyl, wherein at least one of R^{o} or R^{p} is C₁₋₆alkyl. Thus, alkylamino include mono-alkyl amino group (e.g. mono-C₁₋₆alkylamino group such as methylamino and ethylamino), and di-alkylamino group (e.g. di-C₁₋₆alkylamino group such as dimethylamino and diethylamino). Non-limiting examples of suitable mono- or di-C₁₋₆alkylamino groups include n-propylamino, isopropylamino, n-butylamino, i-butylamino, sec-butylamino, t-butylamino, pentylamino, n-hexylamino, di-n-propylamino, di-i-propylamino, ethylmethylamino, methyl-n-propylamino, methyl-i-propylamino, n-butylmethylamino, i-butylmethylamino, t-butylmethylamino, ethyl-n-propylamino, ethyl-i-propylamino, n-butylethylamino, i-butylethylamino, t-butylethylamino, di-n-butylamino, di-i-butylamino, methylpentylamino, methylhexylamino, ethylpentylamino, ethylhexylamino, propylpentylamino, propylhexylamino, and the like.

The present invention includes all possible stereoisomers compounds of general formula (I) and any subgroup thereof and includes not only racemic compounds but the individual enantiomers as well. When a compound is desired as a single enantiomer, such may be obtained by stereospecific synthesis, by resolution of the final product or any convenient intermediate, or by chiral chromatographic methods as each are known in the art. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994), incorporated by reference with regard to stereochemistry.

Where structural isomers are interconvertible via a low energy barrier, tautomeric isomerism (‘tautomerism’) can occur. This can take the form of proton tautomerism in compounds of general formula (I) containing, for example, an imino, keto, or oxime group, or so-called valence tautomerism in compounds which contain an aromatic moiety. It follows that a single compound may exhibit more than one type of isomerism.

Conventional techniques for the preparation/isolation of individual enantiomers include chiral synthesis from a suitable optically pure precursor or resolution of the racemate (or the racemate of a salt or derivative) using, for example, chiral high performance liquid chromatography (HPLC).

The compounds of general formula (I) may be prepared as described in the experimental section below using methods and chemistries with which those skilled in the art shall be familiar.

Generally, the compounds of the invention are prepared from the intermediates described hereinafter which may be reacted with complementary reactive molecules so as to form the desired compound. Included within the scope of the present invention are all stereoisomers, diastereomers, geometric isomers and tautomeric forms of the compounds of general formula (I), including compounds exhibiting more than one type of isomerism, and mixtures of one or more thereof. Also included are acid addition or base salts wherein the counterion is optically active, for example, d-lactate or /-lysine, or racemic, for example, dl-tartrate or dl-arginine.

Since the compounds of general formula (I) contain a vinyl group, geometric cis/trans (or Z/E) isomers are possible. In some embodiments, the E isomers of the vinyl group are preferred.

As used herein and unless otherwise stated, the term “enantiomer” means each individual optically active form of a compound of the invention, having an optical purity or enantiomeric excess (as determined by methods standard in the art) of at least 80% (i.e. at least 90% of one enantiomer and at most 10% of the other enantiomer), preferably at least 90% and more preferably at least 98%.

As used herein and unless otherwise stated, the term “solvate” includes any combination which may be formed by derivative of this invention with a suitable inorganic solvent (e.g. hydrates) or organic solvent, such as but not limited to alcohols, ketones, esters, ethers, nitriles and the like.

Salts, and in particular pharmaceutically acceptable salts of the compounds of general structure (I) include the acid addition and base salts thereof. Suitable acid addition salts are formed from acids which form non-toxic salts. Examples include the acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002), incorporated herein by reference.

Salts, and in particular pharmaceutically acceptable salts of compounds of general structure (I) may be prepared by one or more of these methods:
(i) by reacting the compound of general structure (I) with the desired acid;
(ii) by reacting the compound of general structure (I) with the desired base;
(iii) by removing an acid- or base-labile protecting group from a suitable precursor of the compound of general structure (I) or by ring-opening a suitable cyclic precursor, for example, a lactone or lactam, using the desired acid; or
(iv) by converting one salt of the compound of general structure (I) to another by reaction with an appropriate acid or by means of a suitable ion exchange column.

All these reactions are typically carried out in solution. The salt may precipitate from solution and be collected by filtration or may be recovered by evaporation of the solvent. The degree of ionization in the salt may vary from completely ionized to almost non-ionized.

The salts, and in particular pharmaceutically acceptable salts of the compounds according to the invention, i.e. in the form of water-, oil-soluble, or dispersible products, include the conventional non-toxic salts or the quaternary ammonium salts which are formed, e.g., from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalene-sulfonate, nicotinate, oxalate, palmoate, pectinate, persulfate, 3-phenylpropionate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. In addition, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl-bromides and others. Other pharmaceutically acceptable salts include the sulfate salt ethanolate and sulfate salts.

The term “individual” as used herein refers to a mammal. The individual will preferably be a human, but may also be a domestic livestock, laboratory or pet animals.

In some embodiments, the present invention relates to the use of at least one compound of general structure (I), in (the preparation of a composition for) the treatment and/or prevention of viral infections.

The invention also generally covers all pharmaceutically acceptable prodrugs or “pre-drugs” of the compounds of formula (I) or (II) for which general reference is made to the prior art cited hereinbelow.

The term “pro-drug” as used herein means the pharmacologically acceptable derivatives such as esters, amides and phosphates, such that the resulting in vivo biotransformation product of the derivative is the active drug. The reference by Goodman and Gilman (The Pharmacological Basis of Therapeutics, 8th Ed, McGraw-Hill, Int. Ed. 1992, “Biotransformation of Drugs”, p 13-15) describing pro-drugs generally is hereby incorporated. Pro-drugs of the compounds of the invention can be prepared by modifying functional groups present in said component in such a way that the modifications are cleaved, either in routine manipulation or in vivo, to the parent component. Typical examples of pro-drugs are described for instance in WO 99/33795, WO 99/33815, WO 99/33793 and WO 99/33792 all incorporated herein by reference. Pro-drugs are characterized by increased bio-availability and are readily metabolized into the active inhibitors in vivo. The term “pre-drug”, as used herein, means any compound that will be modified to form a drug species, wherein the modification may take place either inside or outside of the body, and either before or after the pre-drug reaches the area of the body where administration of the drug is indicated.

In some embodiments, the prodrugs of the compounds of general structure (I) are ester derivatives.

In some embodiments, the present invention relates to a method of treatment and/or prevention of viral infections, comprising administering to a subject in need thereof an effective amount of at least one compound of general structure (I), or a pharmaceutical composition comprising said at least one compound of general structure (I).

In some embodiments, the present invention relates to the use of at least one compound of general structure (I), in (the preparation of a composition for) the treatment and/or prevention of viral infections, preferably viral infections caused by an RNA virus, more preferably viral infections caused by coronavirus, measles, tacaribe virus, yellow fever virus, influenzavirus, Chikungunya, dengue, respiratory syncytial virus (RSV), human immunodeficiency virus (HIV) and norovirus.

The invention further provides pharmaceutical compositions that include effective amounts of compounds of general structure (I), or pharmaceutically accepted salts thereof, and at least one pharmaceutically acceptable carrier. The compounds of general structure (I) or pharmaceutically acceptable salts thereof, are as herein described.

The compounds according to the invention may be administered as the sole active ingredient or together, i.e. in a fixed or free combination, with other therapeutic agents used in clinical practice for the treatment of those diseases listed above.

The compounds according to the invention and the other pharmaceutical active agent(s) may be administered together or separately and, when administered separately, administration may occur simultaneously or sequentially, in any order.

The amounts of the compounds according to the invention and the other pharmaceutically active agent(s) and the relative timings of administration will be selected in order to achieve the desired combined therapeutic effect. The administration in combination of a compound of formula (I) or a stereoisomer, tautomer, racemic, salt, hydrate or solvate thereof, with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time.

For pharmaceutical use, the compounds of the invention may be used as a free acid or base, and/or in the form of a pharmaceutically acceptable acid-addition and/or base-addition salt (e.g. obtained with non-toxic organic or inorganic acid or base), in the form of a hydrate, solvate and/or complex, and/or in the form or a pro-drug or pre-drug, such as an ester. As used herein and unless otherwise stated, the term “solvate” includes any combination which may be formed by a compound of this invention with a suitable inorganic solvent (e.g. hydrates) or organic solvent, such as but not limited to alcohols, ketones, esters and the like. Such salts, hydrates, solvates, etc. and the preparation thereof will be clear to the skilled person; reference is for instance made to the salts, hydrates, solvates, etc. described in U.S. Pat. Nos. 6,372,778, 6,369,086, 6,369,087 and 6,372,733.

Generally, for pharmaceutical use, the compounds of the inventions may be formulated as a pharmaceutical preparation comprising at least one compound of the invention and at least one pharmaceutically acceptable carrier, diluent or excipient and/or adjuvant, and optionally one or more further pharmaceutically active compounds.

By means of non-limiting examples, such a formulation may be in a form suitable for oral administration, for parenteral administration (such as by intravenous, intramuscular or subcutaneous injection or intravenous infusion), for topical administration (including ocular), for administration by inhalation, by a skin patch, by an implant, by a suppository, etc. Such suitable administration forms-which may be solid, semi-solid or liquid, depending on the manner of administration-as well as methods and carriers, diluents and excipients for use in the preparation thereof, will be clear to the skilled person; reference is again made to for instance U.S. Pat. Nos. 6,372,778, 6,369,086, 6,369,087 and 6,372,733, as well as to the standard handbooks, such as the latest edition of Remington's Pharmaceutical Sciences.

Some preferred, but non-limiting examples of such preparations include tablets, pills, powders, lozenges, sachets, cachets, elixirs, suspensions, emulsions, solutions, syrups, aerosols, ointments, cremes, lotions, soft and hard gelatin capsules, suppositories, drops, sterile injectable solutions and sterile packaged powders (which are usually reconstituted prior to use) for administration as a bolus and/or for continuous administration, which may be formulated with carriers, excipients, and diluents that are suitable per se for such formulations, such as lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propylhydroxybenzoates, talc, magnesium stearate, edible oils, vegetable oils and mineral oils or suitable mixtures thereof. The formulations can optionally contain other pharmaceutically active substances (which may or may not lead to a synergistic effect with the compounds of the invention) and other substances that are commonly used in pharmaceutical formulations, such as lubricating agents, wetting agents, emulsifying and suspending agents, dispersing agents, disintegrants, bulking agents, fillers, preserving agents, sweetening agents, flavoring agents, flow regulators, release agents, etc. The compositions may also be formulated so as to provide rapid, sustained or delayed release of the active compound(s) contained therein, for example using liposomes or hydrophilic polymeric matrices based on natural gels or synthetic polymers. In order to enhance the solubility and/or the stability of the compounds of a pharmaceutical formulation according to the invention, it can be advantageous to employ α-, β- or γ-cyclodextrins or their derivatives. In addition, co-solvents such as alcohols may improve the solubility and/or the stability of the compounds. In the preparation of aqueous compositions, addition of salts of the compounds of the invention can be more suitable due to their increased water solubility.

Appropriate cyclodextrins are α-, β- or γ-cyclodextrins (CDs) or ethers and mixed ethers thereof wherein one or more of the hydroxyl groups of the anhydroglucose units of the cyclodextrin are substituted with alkyl, particularly methyl, ethyl or isopropyl, e.g. randomly methylated β-CD; hydroxyalkyl, particularly hydroxyethyl, hydroxypropyl or hydroxybutyl; carboxyalkyl, particularly carboxymethyl or carboxyethyl; alkylcarbonyl, acetyl; alkoxycarbonylalkyl or particularly carboxyalkoxyalkyl, particularly carboxymethoxypropyl or carboxyethoxypropyl; alkylcarbonyloxyalkyl, particularly 2-acetyloxypropyl. Especially noteworthy as complexants and/or solubilizers are β-CD, randomly methylated β-CD, 2,6-dimethyl-β-CD, 2-hydroxyethyl-β-CD, 2-hydroxyethyl-γ-CD, 2-hydroxypropyl-γ-CD and (2-carboxymethoxy)propyl-β-CD, and in particular 2-hydroxypropyl-β-CD (2-HP-β-CD). The term mixed ether denotes cyclodextrin derivatives wherein at least two cyclodextrin hydroxyl groups are etherified with different groups such as, for example, hydroxypropyl and hydroxyethyl. An interesting way of formulating the compounds in combination with a cyclodextrin or a derivative thereof has been described in EP-A-721,331. Although the formulations described therein are with antifungal active ingredients, they are equally interesting for formulating the compounds. Said formulations may also be rendered more palatable by adding pharmaceutically acceptable sweeteners and/or flavors. In particular, the present invention encompasses a pharmaceutical formulation comprising an effective amount of a compound according to the invention with a pharmaceutically acceptable cyclodextrin. The present invention also encompasses cyclodextrin complexes consisting of a compound according to the invention and a cyclodextrin.

Particular reference is made to the compositions, formulations (and carriers, excipients, diluents, etc. for use therein), routes of administration etc., which are known per se such as those described in U.S. Pat. No. 4,997,834 and EP-A-0 370 498.

More in particular, the compositions may be formulated in a pharmaceutical formulation comprising a therapeutically effective amount of particles consisting of a solid dispersion of the compounds of the invention and one or more pharmaceutically acceptable water-soluble polymers.

The term “a solid dispersion” defines a system in a solid state (as opposed to a liquid or gaseous state) comprising at least two components, wherein one component is dispersed more or less evenly throughout the other component or components. When said dispersion of the components is such that the system is chemically and physically uniform or homogenous throughout or consists of one phase as defined in thermodynamics, such a solid dispersion is referred to as “a solid solution”. Solid solutions are preferred physical systems because the components therein are usually readily bioavailable to the organisms to which they are administered. The term “a solid dispersion” also comprises dispersions that are less homogenous throughout than solid solutions. Such dispersions are not chemically and physically uniform throughout or comprise more than one phase.

The water-soluble polymer is conveniently a polymer that has an apparent viscosity of 1 to 100 mPa·s when dissolved in a 2% aqueous solution at 20° C. solution.

Preferred water-soluble polymers are hydroxypropyl methylcelluloses or HPMC. HPMC having a methoxy degree of substitution from about 0.8 to about 2.5 and a hydroxypropyl molar substitution from about 0.05 to about 3.0 are generally water soluble. Methoxy degree of substitution refers to the average number of methyl ether groups present per anhydroglucose unit of the cellulose molecule. Hydroxy-propyl molar substitution refers to the average number of moles of propylene oxide which have reacted with each anhydroglucose unit of the cellulose molecule.

It may further be convenient to formulate the compounds in the form of nanoparticles which have a surface modifier adsorbed on the surface thereof in an amount sufficient to maintain an effective average particle size of less than 1000 nm. Suitable surface modifiers can preferably be selected from known organic and inorganic pharmaceutical excipients. Such excipients include various polymers, low molecular weight oligomers, natural products, and surfactants. Preferred surface modifiers include nonionic and anionic surfactants.

Yet another interesting way of formulating the compounds according to the invention involves a pharmaceutical composition whereby the compounds are incorporated in hydrophilic polymers and applying this mixture as a coat film over many small beads, thus yielding a composition with good bio-availability which can conveniently be manufactured and which is suitable for preparing pharmaceutical dosage forms for oral administration. Said beads comprise (a) a central, rounded, or spherical core, (b) a coating film of a hydrophilic polymer and an antiretroviral agent and (c) a seal-coating polymer layer. Materials suitable for use as cores in the beads are manifold, provided that said materials are pharmaceutically acceptable and have appropriate dimensions and firmness. Examples of such materials are polymers, inorganic substances, organic substances, and saccharides, and derivatives thereof.

The preparations may be prepared in a manner known per se, which usually involves mixing the at least one compound according to the invention with the one or more pharmaceutically acceptable carriers, and, if desired, in combination with other pharmaceutical active compounds, when necessary under aseptic conditions. Reference is again made to U.S. Pat. Nos. 6,372,778, 6,369,086, 6,369,087 and 6,372,733 and the further prior art mentioned above, as well as to the standard handbooks, such as the latest edition of Remington's Pharmaceutical Sciences.

The pharmaceutical formulations of the invention are preferably in a unit dosage form, and may be suitably packaged, for example in a box, blister, vial, bottle, sachet, ampoule or in any other suitable single-dose or multi-dose holder or container (which may be properly labelled); optionally with one or more leaflets containing product information and/or instructions for use. Generally, such unit dosages will contain between 1 and 1000 mg, and usually between 5 and 500 mg, of the at least one compound of the invention, e.g. about 10, 25, 50, 100, 200, 300 or 400 mg per unit dosage.

The compounds can be administered by a variety of routes including the oral, ocular, rectal, transdermal, subcutaneous, intravenous, intramuscular or intranasal routes, depending mainly on the specific preparation used and the condition to be treated or prevented, and with oral and intravenous administration usually being preferred. The at least one compound of the invention will generally be administered in an “effective amount”, by which is meant any amount of a compound of the formula (I) above that, upon suitable administration, is sufficient to achieve the desired therapeutic or prophylactic effect in the subject to which it is administered. Usually, depending on the condition to be prevented or treated and the route of administration, such an effective amount will usually be between 0.01 to 1000 mg per kilogram, more often between 0.1 and 500 mg, such as between 1 and 250 mg, for example about 5, 10, 20, 50, 100, 150, 200 or 250 mg, per kilogram body weight day of the patient per day, which may be administered as a single daily dose, divided over one or more daily doses, or essentially continuously, e.g. using a drip infusion. The amount(s) to be administered, the route of administration and the further treatment regimen may be determined by the treating clinician, depending on factors such as the age, gender and general condition of the patient and the nature and severity of the disease/symptoms to be treated. Reference is again made to U.S. Pat. Nos. 6,372,778, 6,369,086, 6,369,087 and 6,372,733 and the further prior art mentioned above, as well as to the standard handbooks, such as the latest edition of Remington's Pharmaceutical Sciences.

In accordance with the method of the present invention, said pharmaceutical formulation can be administered separately at different times during the course of therapy or concurrently in divided or single combination forms. The present invention is therefore to be understood as embracing all such regimes of simultaneous or alternating treatment and the term “administering” is to be interpreted accordingly.

For an oral administration form, the compositions of the present invention can be mixed with suitable additives, such as excipients, stabilizers or inert diluents, and brought by means of the customary methods into the suitable administration forms, such as tablets, coated tablets, hard capsules, aqueous, alcoholic, or oily solutions. Examples of suitable inert carriers are gum arabic, magnesia, magnesium carbonate, potassium phosphate, lactose, glucose, or starch, in particular, corn starch. In this case, the preparation can be carried out both as dry and as moist granules. Suitable oily excipients or solvents are vegetable or animal oils, such as sunflower oil or cod liver oil. Suitable solvents for aqueous or alcoholic solutions are water, ethanol, sugar solutions, or mixtures thereof. Polyethylene glycols and polypropylene glycols are also useful as further auxiliaries for other administration forms. As immediate release tablets, these compositions may contain microcrystalline cellulose, dicalcium phosphate, starch, magnesium stearate and lactose and/or other excipients, binders, extenders, disintegrants, diluents and lubricants known in the art.

When administered by nasal aerosol or inhalation, these compositions may be prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. Suitable pharmaceutical formulations for administration in the form of aerosols or sprays are, for example, solutions, suspensions or emulsions of the compounds of the invention or their physiologically tolerable salts in a pharmaceutically acceptable solvent, such as ethanol or water, or a mixture of such solvents. If required, the formulation can also additionally contain other pharmaceutical auxiliaries such as surfactants, emulsifiers and stabilizers as well as a propellant.

For subcutaneous or intravenous administration, the compound according to the invention, if desired with the substances customary therefore such as solubilizers, emulsifiers or further auxiliaries are brought into solution, suspension, or emulsion. The compounds of the invention can also be lyophilized and the lyophilizates obtained used, for example, for the production of injection or infusion preparations. Suitable solvents are, for example, water, physiological saline solution or alcohols, e.g. ethanol, propanol, glycerol, in addition also sugar solutions such as glucose or mannitol solutions, or alternatively mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, these formulations may be prepared by mixing the compounds according to the invention with a suitable non-irritating excipient, such as cocoa butter, synthetic glyceride esters or polyethylene glycols, which are solid at ordinary temperatures, but liquefy and/or dissolve in the rectal cavity to release the drug.

The compositions are of value in the veterinary field, which for the purposes herein not only includes the prevention and/or treatment of diseases in animals, but also—for economically important animals such as cattle, pigs, sheep, chicken, fish, etc.—enhancing the growth and/or weight of the animal and/or the amount and/or the quality of the meat or other products obtained from the animal. Thus, in a further aspect, the invention relates to a (pharmaceutical) formulation for veterinary use that contains at least one compound of the invention and at least one suitable carrier (i.e. a carrier suitable for veterinary use). The invention also relates to the use of a compound of the invention in the preparation of such a formulation.

The following examples are provided for the purpose of illustrating the present invention and by no means should be interpreted to limit the scope of the present invention.

### EXAMPLES

### 1 Chemistry

The C-ribonucleoside analogue 4.7 prepared according to a literature procedure¹⁹ was used to synthesize 7-substituted-pyrrolo[2,1-f][1,2,4]triazine C-ribonucleoside analogues. Firstly, compound 4.3, which was synthesized according to a literature procedure^{19, 20}, was treated with lithium diisopropylamide at −78° C. and thus converted to organolithium intermediate as a highly active nucleophilic reagent that reacted with 2,3,5-tri-O-benzyl-D-ribonolactone via an addition reaction to yield compound 4.4 as a mixture of α and β isomers in good yield (77%) (Scheme 1).

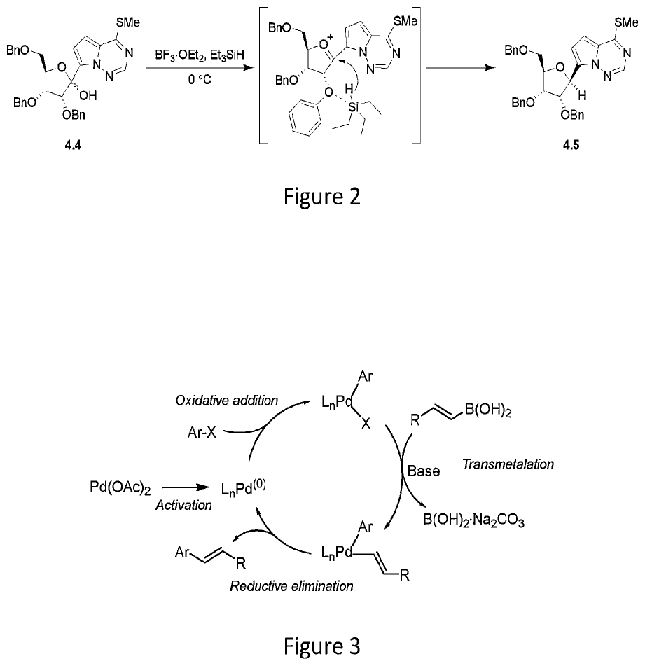

Then, upon treatment with boron trifluoride etherate and triethylsilane, the above mixture was stereoselectively converted to β-product 4.5 via anomeric reduction in 70%.²¹ The Lewis acid catalyses the leaving of the hydroxy group to form oxocarbenium ion. Due to the chelation of silicon to the lone pair on the 2′- or 3′-position oxygen atoms, the hydride anion attacks the C-1′ from α-face to produce β-isomer (FIG. 2). Finally, the thiomethyl group was displaced with an amino group to give compound 4.6 upon treatment with 7 M NH₃ in MeOH at 100° C., followed by deprotection of all benzyl groups to furnish C-ribonucleoside 4.7.

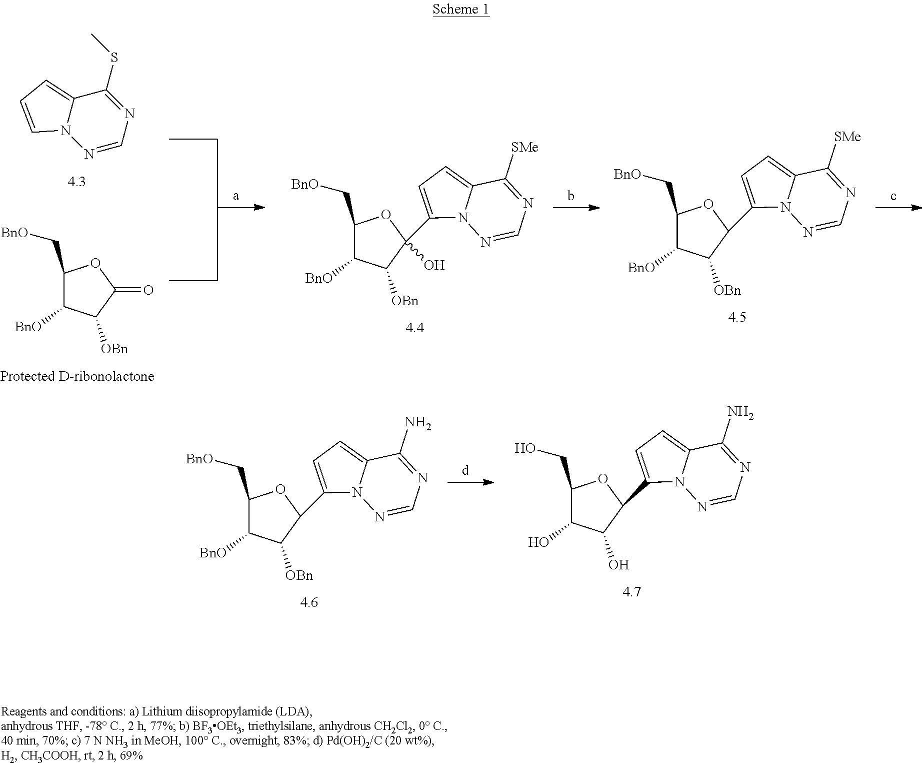 [SMILES: CSc1ncnn2cccc12 ; O=C1O[C@H](COCc2ccccc2)[C@@H](OCc2ccccc2)[C@H]1OCc1ccccc1 ; CSc1ncnn2c(C3(O)O[C@H](COCc4ccccc4)[C@@H](OCc4ccccc4)[C@H]3OCc3ccccc3)ccc12 ; CSc1ncnn2c(C3O[C@H](COCc4ccccc4)[C@@H](OCc4ccccc4)[C@H]3OCc3ccccc3)ccc12 ; Nc1ncnn2c(C3O[C@H](COCc4ccccc4)[C@@H](OCc4ccccc4)[C@H]3OCc3ccccc3)ccc12 ; Nc1ncnn2c([C@@H]3O[C@H](CO)[C@@H](O)[C@H]3O)ccc12]

The key intermediate 4.8 was synthesized from 4.7 via a halogenation and used to introduce different substituents at the 7-position by palladium-catalysed coupling reactions to directly afford derivatives 4.2a-m, including Stille, Heck and Suzuki-Miyaura reactions. The 7-iodo C-ribonucleoside 4.8 was converted compound 4.2a via Stille reaction under treatment with tributyl(vinyl)tin and 5% Pd(PPh₃)₂Cl₂ at 100° C. Meanwhile, compound 4.2b was synthesized upon Heck reaction catalysed by 5% Pd(OAc)₂. Via aqueous Suzuki-Miyaura reaction^{22, 23}, a range of (E)-alkenyl boronic acids (4.9c-4.9m, Scheme 2) were respectively coupled with intermediate 4.8 to obtain 4.2c-4.2m in moderate to good yield (41%-82%). As shown in FIG. 3, after activation of the pre-catalyst Pd(OAc)₂, aryl halide reacted with Pd(0) catalyst via the oxidative addition to Pd(II) intermediate. The boronic acid undergoes the transmetalation in the presence of the base to form new Pd(II) complex. After that, the target product is furnished via the reductive elimination and regenerates the Pd(0) catalyst. Among these (E)-alkenyl boronic acids, boronic acids 4.9h-m were prepared according to a literature procedure^{24, 25}, which were used in the coupling reaction without further purification.

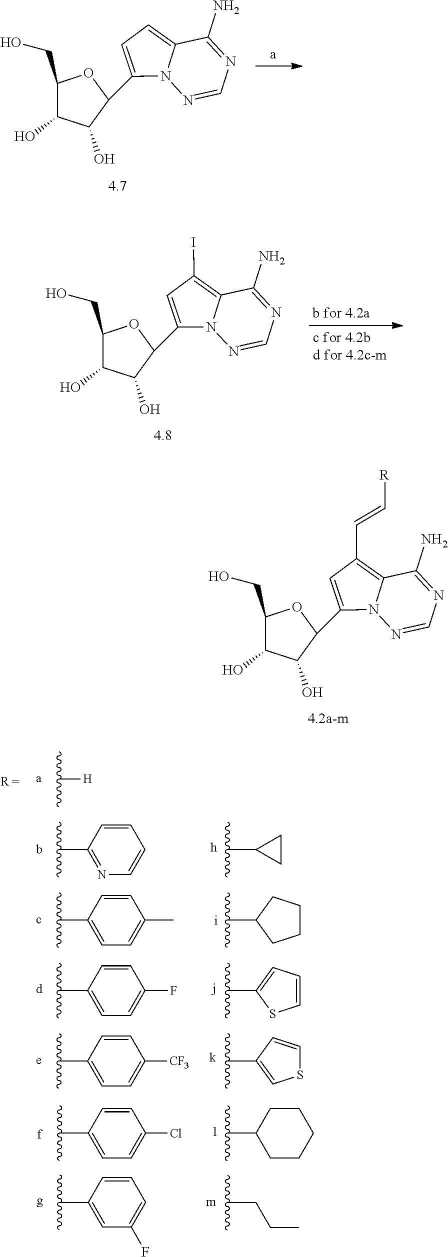

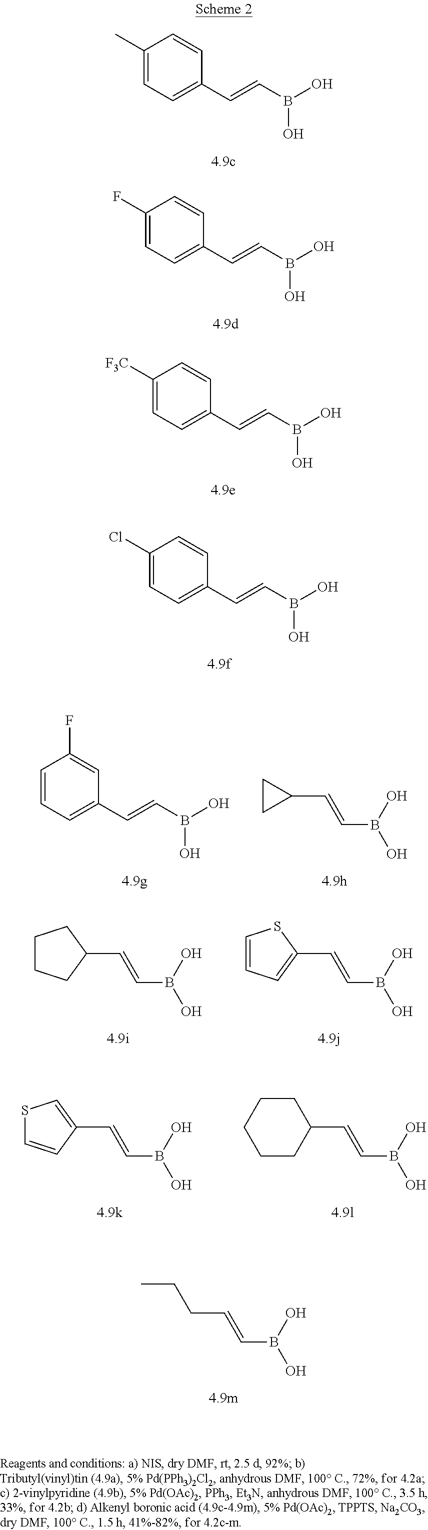

### 2. Antiviral Activity Against SARS-CoV-2

All synthesized C-nucleoside derivatives 4.2a-m were investigated for activity against SARS-CoV-2 and their cytotoxicity. Activity of compounds 4.2a-e against SARS-CoV-2 was evaluated in Vero 76 cells, while protease inhibitor M-128533, a known compound with activity against MERS, was used as positive control. The other compounds 4.2f-m were tested in Vero E6 cells and corresponding positive control was EIDD-1931 (β-D-N⁴-Hydroxycytidine).

The 7-vinyl analogue 4.2a was active against SARS-CoV-2 and accompanied with significant cytotoxicity, showing a moderate selectivity index (Table 1). The introduction of different substituted alkenes (E configuration) at the 7-position was attempted to develop active candidates with high selectivity. Derivative 4.2b bearing pyridyl group displayed potent cytotoxicity. Among analogues 4.2c-e bearing 4-substituted phenyl groups, compound 4.2c showed increased selectivity index (SI=150) as well as potent antiviral activity (EC₅₀=0.3 μg/mL). Interestingly, the replacement of the methyl group with small groups, such as fluorine atom and trifluoro group, exhibited no positive effect on antiviral activity. Although the polar trifluoro group has similar steric hindrance with the methyl group, compound 4.2e bearing CF₃ group was inactive and had similar cytotoxicity with compound 4.2c. It indicates the nonpolar group would be better than polar group at the 4-position of phenyl group. In addition, compounds 4.2f-g bearing halogenated phenyl group and 4.2h-m bearing other alkyl groups and five-member thiophene groups were evaluated in Vero E6 cell assay. Unfortunately, compounds 4.2f-m showed no activity against SARS-CoV-2 (Table 2).

TABLE 1
Activity and cytotoxicity of derivatives 4.2a-e
against SARS-CoV-2 in Vero 76 cells.

| | | EC₅₀ | | |
|---|---|---|---|---|
| | | (mg/mL) | CC₅₀ | |
| Compound | | USA_WA1/ | (mg/mL) | |
| Name | Drug Assay Name | 2020 strain | Vero 76 | SI₅₀ |
| 4.2a | Visual (Cytopathic | <0.1 | 3.2 | >32 |
| | effect/Toxicity) | | | |
| | Neutral red | 0.16 | 4.7 | 29 |
| | (Cytopathic | | | |
| | effect/Toxicity) | | | |
| 4.2b | Visual (Cytopathic | >0.32 | 0.32 | 0 |
| | effect/Toxicity) | | | |
| | Neutral red | <0.1 | 0.32 | >3.2 |
| | (Cytopathic | | | |
| | effect/Toxicity) | | | |
| 4.2c | Visual (Cytopathic | 0.32 | 32 | 100 |
| | effect/Toxicity) | | | |
| | Neutral red | 0.3 | 46 | 150 |
| | (Cytopathic | | | |
| | effect/Toxicity) | | | |
| 4.2d | Visual (Cytopathic | >3.2 | 3.2 | 0 |
| | effect/Toxicity) | | | |
| | Neutral red | >5.8 | 5.8 | 0 |
| | (Cytopathic | | | |
| | effect/Toxicity) | | | |
| 4.2e | Visual (Cytopathic | >32 | 32 | 0 |
| | effect/Toxicity) | | | |
| | Neutral red | >32 | 32 | 0 |
| | (Cytopathic | | | |
| | effect/Toxicity) | | | |
| M- | Visual (Cytopathic | 0.24 | >100 | >420 |
| 128533 | effect/Toxicity) | | | |
| | Neutral red | 0.29 | >100 | >340 |
| | (Cytopathic | | | |
| | effect/Toxicity) | | | |

TABLE 2
Activity and cytotoxicity of derivatives 4.2f-m
against SARS-CoV-2 in Vero E6 cells.

| | | EC₅₀ (mM/mL) | CC₅₀ | |
|---|---|---|---|---|
| Compound | | USA_WA1/2020 | (mM/mL) | |
| Name | Drug Assay Name | strain | Vero E6 | SI₅₀ |
| 4.2f | Visual (Cytopathic | >32 | 32 | 0 |
| | effect/Toxicity) | | | |
| | Neutral red | >32 | 32 | 0 |
| | (Cytopathic | | | |
| | effect/Toxicity) | | | |
| 4.2g | Visual (Cytopathic | >32 | 32 | 0 |
| | effect/Toxicity) | | | |
| | Neutral red | >32 | 32 | 0 |
| | (Cytopathic | | | |
| | effect/Toxicity) | | | |
| 4.2h | Visual (Cytopathic | >100 | >100 | 0 |
| | effect/Toxicity) | | | |
| | Neutral red | >100 | >100 | 0 |
| | (Cytopathic | | | |
| | effect/Toxicity) | | | |
| 4.2i | Visual (Cytopathic | >100 | >100 | 0 |
| | effect/Toxicity) | | | |
| | Neutral red | >100 | >100 | 0 |
| | (Cytopathic | | | |
| | effect/Toxicity) | | | |
| 4.2j | Visual (Cytopathic | >28 | 28 | 0 |
| | effect/Toxicity) | | | |
| | Neutral red | >32 | 32 | 0 |
| | (Cytopathic | | | |
| | effect/Toxicity) | | | |
| 4.2k | Visual (Cytopathic | >3.2 | 3.2 | 0 |
| | effect/Toxicity) | | | |
| | Neutral red | >3.1 | 3.1 | 0 |
| | (Cytopathic | | | |
| | effect/Toxicity) | | | |
| 4.2l | Visual (Cytopathic | >100 | >100 | 0 |
| | effect/Toxicity) | | | |
| | Neutral red | >100 | >100 | 0 |
| | (Cytopathic | | | |
| | effect/Toxicity) | | | |
| 4.2m | Visual (Cytopathic | >100 | >100 | 0 |
| | effect/Toxicity) | | | |
| | Neutral red | >100 | >100 | 0 |
| | (Cytopathic | | | |
| | effect/Toxicity) | | | |
| EIDD- | Visual (Cytopathic | 0.24 | 46 | 190 |
| 1931 | effect/Toxicity) | mg/mL | mg/ml | |
| | Neutral red | 0.26 | 19 | 73 |
| | (Cytopathic | mg/mL | mg/mL | |
| | effect/Toxicity) | | | |

### 3 Experiment Section

### 3.1 General Information

All reagents and solvents were of analytical grade and used without further purification. All sensitive reactions were carried out in dry solvents under an argon or nitrogen atmosphere. ¹H, ¹³C, and ³¹P NMR spectra were obtained on a 300, 500 or 600 MHz Bruker Avance spectrometer using tetramethylsilane (TMS) as an internal standard or by referencing to the residual solvent signal. Coupling constants are reported in hertz (Hz) and were directly obtained from the spectra. The following abbreviations were used to denote the NMR splitting patterns: s (singlet), d (doublet), t (triplet), q (quartet), dd (doublet of doublets), dt (doublet of triplets), m (multiplet), and br (broad). High-resolution mass spectra (HRMS) were obtained on a quadrupole orthogonal acceleration time-of-flight mass spectrometer (Synapt G2, HDMS, Waters, Milford, MA). Samples were infused at 3 μL/min, and spectra were obtained in positive (or negative) ionization mode with a resolution of 15 000 FWHM using leucine enkephalin as the lock mass. Pre-coated aluminum sheets (254 nm) were used for thin layer chromatography (TLC). Intermediate compounds were purified by silica gel column chromatography (60 Å, 0.060-0.200 mm, Acros Organics). Purities of all of the tested compounds were above 95% by HPLC analysis.

### 2′,3′,5′-O-Tribenzyl-1′-hydroxy-4-aza-7,9-dideazaadenosine (4.4)

To a solution of 4.3 (5.92 g, 35.8 mmol) in anhydrous THF (80 mL) was added 2M LDA in THF (23.9 mL, 47.8 mmol) at −78° C. under an argon atmosphere and stirred for 30 min at −78° C. Then, to the mixture was dropwise added fully protected D-ribonolactone (10.0 g, 23.9 mmol) in anhydrous THF (40 mL) at the same temperature. After that, the mixture was stirred for 2 h at −78° C. Then, the reaction was quenched with sat. aqueous NH₄Cl and the mixture was extracted with EtOAc. The combined organic layer was washed with water, brine, dried over with MgSO₄, filtered and concentrated in vacuo. The residue was purified by column chromatography (silica gel, Heptane/EtOAc=4/1, 3/1, 2/1) to afford 4.5 (10.8 g, 77% yield) as yellow syrup. HRMS (ESI⁺): m/z, [M+Na]⁺ calcd for C₃₃H₃₃N₃O₅S₁Na₁⁺, 606.2033; found, 606.2045.

### 2′,3′,5′-O-Tribenzyl-6-methylthio-4-aza-7,9-dideazaadenosine (4.5)

To a solution of 4.4 (10.8 g, 18.5 mmol) in anhydrous CH₂Cl₂ (150 mL) was added triethylsilane (11.8 mL, 74.0 mmol) and BF₃·OEt₂ (6.85 mL,55.5 mmol) at 0° C. under an argon atmosphere and stirred for 30 min at 0° C. Then, the reaction was quenched with sat. NaHCO₃ aqueous and extracted with CH₂Cl₂. The organic layer was washed with water, brine, dried over with MgSO₄, filtered and concentrated in vacuo. The residue was purified by column chromatography (silica gel, Heptane/EtOAc=5/1) to afford 4.5 (7.41 g, 70% yield) as yellow syrup. ¹H NMR (300 MHz, CDCl₃): δ 8.19 (1H, s, H-2), 7.38-7.21 (15H, m, 3×Ph—H), 6.80 (1H, d, J=4.6 Hz, H-8), 6.68 (1H, d, J=4.6 Hz, H-7), 5.70 (1H, d, J=4.2 Hz, H-1′), 4.72 (2H, s, CH₂Ph), 4.66-4.37 (5H, m, 2×CH₂Ph and H-4′), 4.25 (1H, t, J=4.6 Hz, H-2′), 4.12 (1H, dd, J₁=6.5 Hz, J₂=4.9 Hz, H-3′), 3.78 (1H, dd, J₁=10.7 Hz, J₂=3.4 Hz, H-5′a), 3.65 (1H, dd, J₁=10.7 Hz, J₂=4.0 Hz H-5′b), 2.65 (3H, s, SCH₃). ¹³C NMR (75 MHz, CDCl₃): δ 164.5 (C-6), 145.2 (C-2), 138.3, 138.0, 137.9 (Ph—C), 129.3 (C-9), 128.4, 128.3, 128.2, 128.0, 127.8, 127.8, 127.7, 127.7 (Ph—C), 122.3 (C-5), 112.3 (C-8), 102.0 (C-7), 80.7 (C-4′), 79.0 (C-2′), 77.2 (C-3′), 76.1 (C-1′), 73.5 (5′-OCH₂Ph), 72.1 (3′-OCH₂Ph), 71.7 (2′-OCH₂Ph), 69.7 (C-5′), 11.5 (SCH₃). HRMS (ESI⁺): m/z, [M+H]⁺ calcd for C₃₃H₃₄N₃O₄S₁⁺, 568.2264; found, 568.2281.

### 2′,3′,5′-O-Tribenzyl-4-aza-7,9-dideazaadenosine (4.6)

4.5 (3.00 g, 5.28 mmol) was dissolved in 7N NH₃ in MeOH (120 mL) in a sealed flask and then heated to 100° C. The mixture was stirred for 24 h at 100° C. overnight. The mixture was concentrated in vacuo. The residue was purified by column chromatography (silica gel, Heptane/EtOAc=1/2) to afford 4.6 (2.38 g, 83% yield). 1H NMR (300 MHz, CDCl₃): δ 7.92 (1H, s, H-2), 7.42-7.13 (15H, m, 3×Ph-H), 6.68 (1H, d, J=4.5 Hz, H-7), 6.49 (1H, d, J=4.5 Hz, H-8), 5.69 (1H, d, J=4.1 Hz, H-1′), 5.63 (2H, br, NH₂), 4.74 (2H, s, CH₂Ph), 4.65-4.37 (5H, m, 2×CH₂Ph and H-4′), 4.28 (1H, t, J=4.6 Hz, H-2′), 4.13 (1H, dd, J₁=6.6 Hz, J₂=4.9 Hz, H-3′), 3.79 (1H, dd, J₁=10.8 Hz, J₂=3.4 Hz, H-5′a), 3.66 (1H, dd, J₁=10.7 Hz, J₂=4.0 Hz, H-5′b). ¹³C NMR (75 MHz, CDCl₃): δ 155.2 (C-6), 147.1 (C-2), 138.4, 138.1, 138.0 (Ph—C), 129.8 (C-9), 128.4, 128.4, 128.3, 128.0, 127.8, 127.8, 127.7, 127.7 (Ph—C), 114.8 (C-5), 110.7 (C-8), 100.0 (C-7), 80.6 (C-4′), 78.9 (C-2′), 77.2 (C-3′), 76.3 (C-1′), 73.5 (5′-OCH₂Ph), 72.1 (3′-OCH₂Ph), 71.6 (2′-OCH₂Ph), 69.8 (C-5′). HRMS (ESI+): m/z, [M+H]⁺ calcd for C₃₂H₃₃N₄O₄⁺, 537.2496; found, 537.2506.

### 4-Aza-7,9-dideazaadenosine (4.7)

To a solution of 4.6 (2.28 g, 4.24 mmol) in AcOH (90 mL) was added Pd (OH)₂/C (20 wt %) (2.28 g) and the mixture was stirred for 2 h at the room temperature under a hydrogen atmosphere (balloon). Then, the mixture was filtered through a Celite pad and the filtrate was concentrated in vacuo. The residue was purified by column chromatography (silica gel, CH₂Cl₂/MeOH=10/1, 5/1) to afford 4.7 (787 mg, 69% yield). ¹H NMR (300 MHz, DMSO-d₆): δ 7.82 (1H, s, H-2), 7.71 (2H, br, NH₂), 6.84 (1H, d, J=4.4 Hz, H-7), 6.68 (1H, d, J=4.4 Hz, H-8), 5.11 (1H, d, J=6.4 Hz, H-1′), 5.00 (1H, d, J=6.2 Hz, 2′-OH), 4.92 (1H, d, J=5.1 Hz, 3′-OH), 4.81 (1H, t, J=5.7 Hz, 5′-OH), 4.23 (1H, q, J=6.1 Hz, H-2′), 3.95 (1H, q, J=5.0 Hz, H-3′), 3.79 (1H, q, J=4.4 Hz, H-4′), 3.59-3.42 (2H, m, H-5′). ¹³C NMR (75 MHz, DMSO-d₆): δ 155.6 (C-6), 147.7 (C-2), 128.9 (C-9), 114.9 (C-5), 109.8 (C-8), 100.8 (C-7), 84.5 (C-4′), 75.4 (C-1′), 73.8 (C-2′), 71.3 (C-3′), 62.1 (C-5′). HRMS (ESI⁺): m/z, [M+H]⁺ calcd for C₁₁H₁₅N₄O₄⁺, 267.1087; found, 267.1091.

### 7-Iodo-4-aza-7,9-dideazaadenosine (4.8)

To a solution of 4.7 (737 mg, 2.76 mmol) in anhydrous DMF (20 mL) was added dropwise NIS (746 mg, 3.32 mmol) in anhydrous DMF (8 mL) in an ice-water bath and stirred for 2.5 d at room temperature. After removing the solvent, the mix-solvent CH₂Cl₂/MeOH (15/1, 12 mL) was added. The suspension was stirred for 2 h, filtered, washed with CH₂Cl₂/MeOH (15/1, 10 mL) and dried to afford 4.8 (1.00 g, 92% yield) as a light-yellow solid. ¹H NMR (300 MHz, DMSO-d₆): δ 7.88 (1H, s, H-2), 6.97 (1H, s, H-8), 5.10 (1H, d, J=6.2 Hz, H-1′), 5.06 (1H, d, J=6.1 Hz, 2′-OH), 4.92 (1H, d, J=5.2 Hz, 3′-OH), 4.79 (1H, d, J=5.7 Hz, 5′-OH), 4.15 (1H, q, J=5.8 Hz, H-2′), 3.93 (1H, q, J=5.0 Hz, H-3′), 3.77 (1H, q, J=3.9 Hz, H-4′), 3.57-3.36 (2H, m, H-5′). ¹³C NMR (75 MHz, DMSO-d₆): δ 155.5 (C-6), 147.8 (C-2), 131.9 (C-9), 118.1 (C-8), 113.9 (C-5), 84.4 (C-4′), 74.9 (C-1′), 74.0 (C-2′), 71.0 (C-3′), 61.7 (C-5′), 52.6 (C-7). HRMS (ESI⁺): m/z, [M+H]⁺ calcd for C₁₁H₁₄I₁N₄O₄⁺, 393.0056; found, 393.0051.

### 7-Vinyl-4-aza-7,9-dideazaadenosine (4.2a)

To a N₂-purged solution of 4.8 (50 mg, 0.127 mmol) and Pd(PPh₃)₂Cl₂ (4.5 mg, 0.006 mmol) in anhydrous DMF (3 mL) was added tributyl(vinyl)tin (42 μL, 0.140 mmol). The mixture was stirred at 100° C. overnight. The mixture was concentrated in vacuo and the residue was purified by column chromatography (silica gel, CH₂Cl₂/MeOH=15/1, 10/1) to afford 4.2a (27 mg, 72% yield) as a white solid. ¹H NMR (300 MHz, DMSO-d₆): δ 7.80 (1H, s, H-2), 7.38 (2H, br, NH₂), 7.25 (1H, dd, J₁=16.9 Hz, J₂=10.8 Hz, vinyl-CH), 7.03 (1H, s, H-8), 5.65 (1H, d, J=17.0 Hz, vinyl-CH₂a), 5.17-5.09 (2H, m, vinyl-CH₂b and H-1′), 5.02 (1H, d, J=6.0 Hz, 2′-OH), 4.91 (1H, d, J=5.2 Hz, 3′-OH), 4.79 (1H, t, J=5.7 Hz, 5′-OH), 4.21 (1H, q, J=6.0 Hz, H-2′), 3.95 (1H, q, J=5.0 Hz, H-3′), 3.79 (1H, q, J=3.8 Hz, H-4′), 3.60-3.41 (2H, m, H-5′). ¹³C NMR (75 MHz, DMSO-d₆): δ 156.0 (C-6), 147.3 (C-2), 130.0 (C-9), 128.8 (vinyl-CH), 115.2 (C-7), 113.1 (vinyl-CH2), 111.5 (C-5), 106.9 (C-8), 84.5 (C-4′), 75.2 (C-1′), 73.8 (C-2′), 71.1 (C-3′), 61.9 (C-5′). HRMS (ESI⁺): m/z, [M+H]⁺ calcd for C₁₃H₁₇N₄O₄⁺, 293.1244; found, 293.1242.

### 7-((E)-2-(Pyridin-2-yl) vinyl)-4-aza-7,9-dideazaadenosine (4.2b)

To an argon-purged mixture of 4.8 (100 mg, 0.255 mmol), 2-vinylpyridine (0.04 mL, 0.382 mmol), PPh₃ (6.7 mg, 0.026 mmol), Pd(OAc)₂ (2.9 mg, 0.013 mmol) and Et₃N (0.12 mL, 0.870 mmol) in anhydrous DMF (6 mL) was stirred for 3.5 h at 100° C. The mixture was concentrated in vacuo and the residue was purified by column chromatography (silica gel, CH₂Cl₂/MeOH=15/1, 10/1) to afford a yellow solid. Then, EtOAc (2 mL) was added and the suspension stirred at the room temperature overnight. The suspension was filtered to obtain 4.2b (32 mg, 33% yield) as a yellow solid. ¹H NMR (300 MHz, DMSO-d₆): δ 8.53 (1H, d, J=4.2 Hz, Ar—H), 7.93-7.69 (4H, m, vinyl-CH, Ar—H and H-2), 7.56 (2H, br, NH₂), 7.27 (1H, s, H-8), 7.20 (1H, dd, J₁=7.4 Hz, J₂=4.7 Hz, Ar—H), 7.14 (1H, d, J=15.8 Hz, vinyl-CH), 5.14 (1H, d, J=6.1 Hz, H-1′), 5.07 (1H, d J=6.1 Hz, 2′-OH), 4.93 (1H, d, J=5.3 Hz, 3′-OH), 4.84 (1H, t, J=5.8 Hz, 5′-OH), 4.25 (1H, q, J=5.9 Hz, H-2′), 3.98 (1H, q, J=5.1 Hz, H-3′), 3.81 (1H, q, J=4.3 Hz, H-4′), 3.63-3.44 (2H, m, H-5′). ¹³C NMR (75 MHz, DMSO-d₆): δ 156.2 (C-6), 156.0 (Ar—C), 149.2 (Ar—C), 147.5 (C-2), 136.3 (Ar—C), 130.7 (C-9), 128.1 (vinyl-C), 124.0 (vinyl-C), 121.6 (Ar—C), 120.9 (Ar—C), 114.5 (C-7), 112.6 (C-5), 107.6 (C-8), 84.4 (C-4′), 75.2 (C-1′), 74.0 (C-2′), 71.1 (C-3′), 61.8 (C-5′). HRMS (ESI⁺): m/z, [M+H]⁺ calcd for C₁₈H₂₀N₅O₄⁺, 370.1509; found, 370.1506.

### 7-((E)-4-Methylstyryl)-4-aza-7,9-dideazaadnosine (4.2c)

An argon-purged mixture of 4.8 (50 mg, 0.127 mmol), trans-2-(4-methylphenyl)vinylboronic acid (31 mg, 0.190 mmol), Na₂CO₃ (30 mg, 0.285 mmol), TPPTS (7.2 mg, 0.012 mmol) and Pd(OAc)₂ (1.5 mg, 0.006 mmol) in H₂O/MeCN (2/1, 3 mL) was heated to 100° C. and stirred for 1.5 h at the same temperature. The mixture was neutralized with 1N HCl (aq) and concentrated in vacuo. The residue was purified by column chromatography (silica gel, CH₂Cl₂/MeOH=15/1, 10/1) to afford 4.2c (20 mg, 41% yield) as a yellow solid. ¹H NMR (600 MHz, MeOD): δ 7.74 (1H, s, H-2), 7.48 (2H, d, J=7.2 Hz, Ar—H), 7.41 (1H, d, J=15.8 Hz, vinyl-CH), 7.15 (2H, d, J=7.9 Hz, Ar—H), 7.11 (1H, s, H-8), 7.04 (1H, d, J=15.9 Hz, vinyl-CH), 5.27 (1H, d, J=6.4 Hz, H-1′), 4.48 (1H, dd, J₁=6.4 Hz, J₂=5.3 Hz, H-2′), 4.20-4.18 (1H, m, H-3′), 4.05 (1H, q, J=4.0 Hz, H-4′), 3.83 (1H, dd, J₁=12.1 Hz, J₂=3.2 Hz, H-5′a), 3.72 (1H, dd, J₁=12.1 Hz, J₂=4.0 Hz, H-5′b), 2.33 (3H, s, CH₃). ¹³C NMR (151 MHz, MeOD): δ 157.9 (C-6), 147.8 (C-2), 138.4 (Ar—C), 136.1 (Ar—C), 131.2 (vinyl-C), 131.1 (C-9), 130.2 (Ar—C), 127.5 (Ar—C), 119.5 (vinyl-C), 118.7 (C-7), 113.4 (C-5), 109.5 (C-8), 86.3 (C-4′), 78.5 (C-1′), 75.2 (C-2′), 73.1 (C-3′), 63.7 (C-5′), 21.3 (CH₃). HRMS (ESI⁺): m/z, [M+H]⁺ calcd for C₂₀H₂₃N₄O₄⁺, 383.1713; found, 383.1712.

### 7-((E)-4-Fluorostyryl)-4-aza-7,9-dideazaadenosine (4.2d)

An argon-purged mixture of 4.8 (50 mg, 0.127 mmol), trans-2-(4-fluorophenyl)vinylboronic acid (32 mg, 0.190 mmol), Na₂CO₃ (30 mg, 0.285 mmol), TPPTS (7.2 mg, 0.012 mmol) and Pd(OAc)₂ (1.5 mg, 0.006 mmol) in H₂O/MeCN (2/1, 3 mL) was heated to 100° C. and stirred for 1.5 h at the same temperature. The mixture was neutralized with 1N HCl(aq) and concentrated in vacuo. The residue was purified by column chromatography (silica gel, CH₂Cl₂/MeOH=15/1, 10/1) to afford 4.2d (22 mg, 44% yield) as a yellow solid. ¹H NMR (600 MHz, MeOD): δ 7.75 (1H, s, H-2), 7.65-7.59 (2H, m, Ar—H), 7.42 (1H, d, J=15.8 Hz, vinyl-CH), 7.13 (1H, s, H-8), 7.09-7.05 (3H, m, vinyl-CH and Ar—H), 5.27 (1H, d, J=6.4 Hz, H-1′), 4.48 (1H, dd, J₁=6.4 Hz, J₂=5.3 Hz, H-2′), 4.20-4.18 (1H, m, H-3′), 4.05 (1H, q, J=4.1 Hz, H-4′), 3.83 (1H, dd, J₁=12.1 Hz, J₂=3.2 Hz, H-5′a), 3.72 (1H, dd, J₁=12.1 Hz, J₂=4.0 Hz, H-5′b). ¹³C NMR (151 MHz, MeOD): δ 164.4, 162.8 (Ar—C), 157.9 (C-6), 147.9 (C-2), 135.4 (Ar—C), 131.2 (C-9), 129.8 (vinyl-C), 129.3, 129.2 (Ar—C), 120.4 (vinyl-C), 118.4 (C-7), 116.4, 116.2 (Ar—C), 113.6 (C-5), 109.5 (C-8), 86.3 (C-4′), 78.5 (C-1′), 75.2 (C-2′), 73.1 (C-3′), 63.7 (C-5′). HRMS (ESI⁺): m/z, [M+H]⁺ calcd for C₁₉H₂₀F₁N₄O₄⁺, 387.1462; found, 387.1457.

### 7-((E)-4-(Trifluoromethyl)styryl)-4-aza-7,9-dideazaadenosine (4.2e)

An argon-purged mixture of 4.8 (50 mg, 0.127 mmol), trans-2-(4-(trifluoromethyl)phenyl)vinylboronic acid (41 mg, 0.190 mmol), Na₂CO₃ (30 mg, 0.285 mmol), TPPTS (7.2 mg, 0.012 mmol) and Pd(OAc)₂ (1.5 mg, 0.006 mmol) in H₂O/MeCN (2/1, 3 mL) was heated to 100° C. and stirred for 1.5 h at the same temperature. The mixture was neutralized with 1N HCl(aq) and concentrated in vacuo. The residue was purified by column chromatography (silica gel, CH₂Cl₂/MeOH=15/1, 10/1) to afford 4.2e (25 mg, 45% yield) as a yellow solid. ¹H NMR (600 MHz, MeOD): δ 7.77-7.74 (3H, m, Ar—H and H-2), 7.62-7.59 (3H, m, Ar—H and vinyl-CH), 7.19 (1H, s, C-8), 7.12 (1H, d, J=15.7 Hz, vinyl-CH), 5.28 (1H, d, J=6.4 Hz, H-1′), 4.49 (1H, dd, J₁=6.4 Hz, J₂=5.3 Hz, H-2′), 4.21-4.18 (1H, m, H-3′), 4.05 (1H, q, J=4.1 Hz, H-4′), 3.84 (1H, dd, J₁=12.1 Hz, J₂=3.2 Hz, H-5′a), 3.73 (1H, dd, J₁=12.1 Hz, J₂=4.0 Hz, H-5′b). ¹³C NMR (151 MHz, MeOD): δ 157.8 (C-6), 148.0 (C-2), 142.8 (Ar—C), 131.4 (C-9), 129.8, 129.6 (Ar—C), 128.9 (vinyl-C), 127.8 (Ar—C), 126.8 (CF₃), 126.4, 126.4 (Ar—C), 125.0 (CF₃), 123.3 (vinyl-C), 117.7 (C-7), 114.0 (C-5), 109.7 (C-8), 86.3 (C-4′), 78.4 (C-1′), 75.3 (C-2′), 73.1 (C-3′), 63.6 (C-5′). HRMS (ESI⁺): m/z, [M+H]⁺ calcd for C₂₀H₂₀F₃N₄O₄⁺, 437.1431; found, 437.1426.

### 7-((E)-4-Chlorostyryl)-4-aza-7,9-dideazaadenosine (4.2f)

An argon-purged mixture of 4.8 (50 mg, 0.127 mmol), trans-2-(4-chlorophenyl)vinylboronic acid (35 mg, 0.190 mmol), Na₂CO₃ (30 mg, 0.285 mmol), TPPTS (7.2 mg, 0.012 mmol) and Pd(OAc)₂ (1.5 mg, 0.006 mmol) in H₂O/MeCN (2/1, 3 mL) was heated to 100° C. and stirred for 1.5 h at the same temperature. The mixture was neutralized with 1N HCl(aq) and concentrated in vacuo. The residue was purified by column chromatography (silica gel, CH₂Cl₂/MeOH=15/1, 10/1) to afford 4.2f (30 mg, 58% yield). ¹H NMR (600 MHz, DMSO-d₆): δ 7.83 (1H, s, H-2), 7.74 (2H, d, J=8.6 Hz, Ar—H), 7.61 (1H, d, J=15.7 Hz, vinyl-CH), 7.58 (2H, br, NH₂), 7.40 (2H, d, J=8.6 Hz, Ar—H), 7.20 (1H, s, H-8), 7.09 (1H, d, J=15.7 Hz, vinyl-CH), 5.13 (1H, d, J=6.3 Hz, H-1′), 5.04 (1H, d, J=6.2 Hz, 2′-OH), 4.93 (1H, d, J=5.3 Hz, 3′-OH), 4.81 (1H, t, J=5.8 Hz, 5′-OH), 4.24 (1H, q, J=6.0 Hz, H-2′), 3.97 (1H, q, J=5.1 Hz, H-3′), 3.81 (1H, q, J=4.5 Hz, H-4′), 3.58 (1H, ddd, J₁=11.7 Hz, J₂=5.4 Hz, J₃=4.0 Hz, H-5′a), 3.51-3.47 (1H, m, H-5′b). ¹³C NMR (151 MHz, DMSO-d₆): δ 156.0 (C-6), 147.5 (C-2), 136.5, 131.2 (Ar—C), 130.4 (C-9), 128.4, 128.2 (Ar—C), 126.6 (vinyl-C), 120.9 (vinyl-C), 115.0 (C-7), 112.1 (C-5), 107.2 (C-8), 84.5 (C-4′), 75.2 (C-1′), 73.9 (C-2′), 71.2 (C-3′), 62.0 (C-5′). HRMS (ESI⁺): m/z, [M+H]⁺ calcd for C₁₉H₂₀Cl₁N₄O₄⁺, 403.1167; found, 403.1164.

### 7-((E)-3-Fluorostyryl)-4-aza-7,9-dideazaadenosine (4.2g)

An argon-purged mixture of 4.8 (50 mg, 0.127 mmol), trans-2-(3-fluorophenyl)vinylboronic acid (32 mg, 0.190 mmol), Na₂CO₃ (30 mg, 0.285 mmol), TPPTS (7.2 mg, 0.012 mmol) and Pd(OAc)₂ (1.5 mg, 0.006 mmol) in H₂O/MeCN (2/1, 3 mL) was heated to 100° C. and stirred for 1.5 h at the same temperature. The mixture was neutralized with 1N HCl(aq) and concentrated in vacuo. The residue was purified by column chromatography (silica gel, CH₂Cl₂/MeOH=15/1, 10/1) to afford 4.2g (27 mg, 55% yield). ¹H NMR (600 MHz, MeOD): δ 7.77 (1H, s, H-2), 7.54 (1H, d, J=15.8 Hz, vinyl-CH), 4.45-4.42 (1H, m, Ar—H), 7.40-7.37 (1H, m, Ar—H), 7.34 (1H, td, J₁=7.9 Hz, J₂=5.9 Hz, Ar—H), 7.17 (1H, s, H-8), 7.09 (1H, d, J=15.7 Hz, vinyl-CH), 6.97-6.93 (1H, m, Ar—H), 5.28 (1H, d, J=6.4 Hz, H-1′), 4.48 (1H, dd, J₁=6.3 Hz, J₂=5.4 Hz, H-2′), 4.20-4.18 (1H, m, H-3′), 4.04 (1H, q, J=4.1 Hz, H-4′), 3.83 (1H, dd, J₁=12.1 Hz, J₂=3.2 Hz, H-5′a), 3.72 (1H, dd, J₁=12.1 Hz, J₂=4.0 Hz, H-5′b). ¹³C NMR (151 MHz, MeOD): δ 165.5, 163.9 (Ar—C), 157.9 (C-6), 147.9 (C-2), 141.6, 141.5, 131.3, 131.2 (Ar—C), 131.2 (C-9), 129.6 (vinyl-C), 123.8 (Ar—C), 122.0 (vinyl-C), 117.9 (C-7), 114.9, 114.8 (Ar—C), 113.8 (C-5), 113.5, 113.4 (Ar—C), 109.6 (C-8), 86.2 (C-4′), 78.5 (C-1′), 75.3 (C-2′), 73.1 (C-3′), 63.6 (C-5′). HRMS (ESI⁺): m/z, [M+H]⁺ calcd for C₁₉H₂₀F₁N₄O₄⁺, 387.1462; found, 387.1460.

### 7-((E)-2-Cyclopropylvinyl)-4-aza-7,9-dideazaadenosine (4.2h)

An argon-purged mixture of 4.8 (50 mg, 0.127 mmol), (E)-2-cyclopropylvinylboronic acid (22 mg, 0.190 mmol), Na₂CO₃ (30 mg, 0.285 mmol), TPPTS (7.2 mg, 0.012 mmol) and Pd(OAc)₂ (1.5 mg, 0.006 mmol) in H₂O/MeCN (2/1, 3 mL) was heated to 100° C. and stirred for 1.5 h at the same temperature. The mixture was neutralized with 1N HCl(aq) and concentrated in vacuo. The residue was purified by column chromatography (silica gel, CH₂Cl₂/MeOH=15/1, 10/1) to afford 4.2h (35 mg, 82% yield). ¹H NMR (300 MHz, MeOD): δ 7.67 (1H, s, H-2), 8.83-7.76 (2H, m, C-8 and vinyl-CH), 5.62 (1H, dd, J₁=15.2 Hz, J₂=9.2 Hz, vinyl-CH), 5.21 (1H, d, J=6.5 Hz, H-1′), 4.45 (1H, t, J=5.8 Hz, H-2′), 4.16 (1H, t, J=4.6 Hz, H-3′), 4.03 (1H, q, J=3.3 Hz, H-4′), 3.80 (1H, dd, J₁=12.1 Hz, J₂=2.6 Hz, H-5′a), 3.69 (1H, dd, J₁=12.2 Hz, J₂=3.6 Hz, H-5′b), 1.70-1.58 (1H, m, CH), 0.89-0.73 (2H, m, CH₂), 0.57-0.42 (2H, m, CH₂). ¹³C NMR (75 MHz, MeOD): δ 157.8 (C-6), 147.5 (C-2), 138.2 (vinyl-C), 130.4 (C-9), 119.5 (vinyl-C), 119.0 (C-7), 112.2 (C-5), 109.5 (C-8), 86.3 (C-4′), 78.4 (C-1′), 75.1 (C-2′), 73.2 (C-3′), 63.7 (C-5′), 15.5 (CH), 7.6 ((CH₂)₂). HRMS (ESI⁺): m/z, [M+H]⁺ calcd for C₁₆H₂₁N₄O₄⁺, 333.1557; found, 333.1552.

### 7-((E)-2-Cyclopentylvinyl)-4-aza-7,9-dideazaadenosine (4.2i)

An argon-purged mixture of 4.8 (50 mg, 0.127 mmol), (E)-2-cyclopentylvinylboronic acid (27 mg, 0.190 mmol), Na₂CO₃ (30 mg, 0.285 mmol), TPPTS (7.2 mg, 0.012 mmol) and Pd(OAc)₂ (1.5 mg, 0.006 mmol) in H₂O/MeCN (2/1, 3 mL) was heated to 100° C. and stirred for 1.5 h at the same temperature. The mixture was neutralized with 1N HCl(aq) and concentrated in vacuo. The residue was purified by column chromatography (silica gel, CH₂Cl₂/MeOH=15/1, 10/1) to afford 4.2i (34 mg, 74% yield). ¹H NMR (600 MHz, MeOD): δ 7.69 (1H, s, H-2), 6.88 (1H, s, H-8), 6.71 (1H, d, J=15.4 Hz, vinyl-CH), 6.10 (1H, dd, J₁=15.3 Hz, J₂=8.4 Hz, vinyl-CH), 5.23 (1H, d, J=5.6 Hz, H-1′), 4.45 (1H, dd, J₁=6.6 Hz, J₂=5.3 Hz, H-2′), 4.17 (1H, dd, J₁=5.3 Hz, J₂=4.3 Hz, H-3′), 4.03 (1H, q, J=4.0 Hz, H-4′), 3.80 (1H, dd, J₁=12.1 Hz, J₂=3.2 Hz, H-5′a), 3.69 (1H, dd, J₁=12.1 Hz, J₂=4.0 Hz, H-5′b), 2.71-2.63 (1H, m, CH), 1.91-1.85 (2H, m, CH₂), 1.77-1.70 (2H, m, CH₂), 1.68-1.59 (2H, m, CH₂), 1.45-1.37 (2H, m, CH₂). ¹³C NMR (151 MHz, MeOD): δ 157.8 (C-6), 147.6 (C-2), 139.0 (vinyl-C), 130.5 (vinyl-C), 120.5 (C-9), 118.8 (C-7), 112.5 (C-5), 109.9 (C-8), 86.3 (C-4′), 78.4 (C-1′), 75.2 (C-2′), 73.2 (C-3′), 63.7 (C-5′), 45.3 (CH), 34.4 (CH₂×2), 26.3 (CH₂×2). HRMS (ESI⁺): m/z, [M+H]⁺ calcd for C₁₈H₂₅N₄O₄⁺, 361.1870; found, 361.1863.

### 7-((E)-2-(Thiophen-2-yl)vinyl)-4-aza-7,9-dideazaadenosine (4.2j)

An argon-purged mixture of 4.8 (50 mg, 0.127 mmol), (E)-(2-(thiophen-2-yl)vinyl)boronic acid (30 mg, 0.190 mmol), Na₂CO₃ (30 mg, 0.285 mmol), TPPTS (7.2 mg, 0.012 mmol) and Pd(OAc)₂ (1.5 mg, 0.006 mmol) in H₂O/MeCN (2/1, 3 mL) was heated to 100° C. and stirred for 1.5 h at the same temperature. The mixture was neutralized with 1N HCl(aq) and concentrated in vacuo. The residue was purified by column chromatography (silica gel, CH₂Cl₂/MeOH=15/1, 10/1) to afford 4.2j (30 mg, 63% yield). ¹H NMR (600 MHz, DMSO-d₆): δ 7.82 (1H, s, H-2), 7.50 (2H, br, NH₂), 7.42 (1H, dd, J₁=5.1 Hz, J₂=1.0 Hz, Ar—H), 7.38 (1H, d, J=15.6 Hz, vinyl-CH), 7.33 (1H, d, J=3.4 Hz, (Ar—H)), 7.24 (1H, d, J=15.5 Hz, vinyl-CH), 7.16 (1H, s, H-8), 7.06 (1H, dd, J₁=5.1 Hz, J₂=3.5 Hz, Ar—H), 5.12 (1H, d, J=6.2 Hz, H-1′), 5.02 (1H, d, J=6.2 Hz, 2′-OH), 4.91 (1H, d, J=5.3 Hz, 3′-OH), 4.79 (1H, t, J=5.8 Hz, 5′-OH), 4.23 (1H, q, J=6.0 Hz, H-2′), 3.97 (1H, q, J=5.1 Hz, H-3′), 3.80 (1H, q, J=4.5 Hz, H-4′), 3.58 (1H, ddd, J₁=11.7 Hz, J₂=5.4 Hz, J₃=4.0 Hz, H-5′a), 3.48 (1H, ddd, J₁=11.5 Hz, J₂=6.2 Hz, J₃=4.8 Hz, H-5′b). ¹³C NMR (151 MHz, DMSO-d₆): δ 156.2 (C-6), 147.4 (C-2), 142.5 (Ar—C), 130.4 (C-9), 127.8, 125.2, 124.7 (Ar—H), 121.0 (vinyl-C), 119.7 (vinyl-C), 115.0 (C-7), 111.9 (C-5), 107.1 (C-8), 84.5 (C-4′), 75.2 (C-1′), 73.9 (C-2′), 71.2 (C-3′), 62.0 (C-5′). HRMS (ESI⁺): m/z, [M+H]⁺ calcd for C₁₇H₁₉N₄O₄S₁⁺, 375.1121; found, 375.1123.

### 7-((E)-2-(Thiophen-3-yl)vinyl)-4-aza-7,9-dideazaadenosine (4.2k)

An argon-purged mixture of 4.8 (50 mg, 0.127 mmol), (E)-(2-(thiophen-3-yl)vinyl)boronic acid (30 mg, 0.190 mmol), Na₂CO₃ (30 mg, 0.285 mmol), TPPTS (7.2 mg, 0.012 mmol) and Pd(OAc)₂ (1.5 mg, 0.006 mmol) in H₂O/MeCN (2/1, 3 mL) was heated to 100° C. and stirred for 1.5 h at the same temperature. The mixture was neutralized with 1N HCl(aq) and concentrated in vacuo. The residue was purified by column chromatography (silica gel, DCM/MeOH=15/1, 10/1) to afford 4.2k (34 mg, 71%). ¹H NMR (600 MHz, DMSO-d₆): δ 7.81 (1H, s, H-2), 7.71-7.69 (1H, m, Ar—H), 7.55-7.53 (2H, m, Ar—H), 7.51 (2H, br, NH₂), 7.43 (1H, d, J=15.7 Hz, vinyl-CH), 7.14-7.10 (2H, m, H-8 and vinyl-CH), 5.13 (1H, d, J=6.3 Hz, H-1′), 5.02 (1H, d, J=6.2 Hz, 2′-OH), 4.90 (1H, d, J=5.3 Hz, 3′-OH), 4.79 (1H, t, J=5.8 Hz, 5′-OH), 4.23 (1H, q, J=6.0 Hz, H-2′), 3.97 (1H, q, J=5.1 Hz, H-3′), 3.81 (1H, q, J=4.5 Hz, H-4′), 3.58 (1H, ddd, J₁=11.7 Hz, J₂=5.4 Hz, J₃=4.0 Hz, H-5′a), 3.51-3.46 (1H, m, H-5′b). ¹³C NMR (151 MHz, DMSO-d₆): δ 156.0 (C-6), 147.4 (C-2), 140.5 (Ar—C), 130.3 (C-9), 126.3, 126.1 (Ar—C), 122.7 (vinyl-C), 122.1 (Ar—C), 119.8 (vinyl-C), 115.3 (C-7), 111.7 (C-5), 106.7 (C-8), 84.5 (C-4′), 75.2 (C-1′), 73.9 (C-2′), 71.2 (C-3′), 62.0 (C-5′). HRMS (ESI⁺): m/z, [M+H]⁺ calcd for C₁₇H₁₉N₄O₄S₁⁺, 375.1121; found, 375.1122.

### 7-((E)-2-Cyclohexylvinyl)-4-aza-7,9-dideazaadenosine (4.2l)

An argon-purged mixture of 4.8 (50 mg, 0.127 mmol), (E)-(2-cyclohexylvinyl)boronic acid (30 mg, 0.190 mmol), Na₂CO₃ (30 mg, 0.285 mmol), TPPTS (7.2 mg, 0.012 mmol) and Pd(OAc)₂ (1.5 mg, 0.006 mmol) in H₂O/MeCN (2/1, 3 mL) was heated to 100° C. and stirred for 1.5 h at the same temperature. The mixture was neutralized with 1N HCl(aq) and concentrated in vacuo. The residue was purified by column chromatography (silica gel, CH₂Cl₂/MeOH=15/1, 10/1) to afford 4.21 (20 mg, 42% yield). ¹H NMR (600 MHz, MeOD): δ 7.69 (1H, s, H-2), 6.86 (1H, s, H-8), 6.69 (1H, d, J=15.4 Hz, vinyl-CH), 6.07 (1H, dd, J₁=15.5 Hz, J₂=7.7 Hz, vinyl-CH), 5.23 (1H, d, J=6.5 Hz, H-1′), 4.45 (1H, dd, J₁=6.5 Hz, J₂=5.3 Hz, H-2′), 4.18-4.15 (1H, m, H-3′), 4.02 (1H, q, J=3.8 Hz, H-4′), 3.80 (1H, dd, J₁=12.1 Hz, J₂=3.2 Hz, H-5′a), 3.69 (1H, dd, J₁=12.1 Hz, J₂=4.0 Hz, H-5′b), 2.24-2.17 (1H, m, CH), 1.84-1.76 (4H, m, CH₂×2), 1.72-1.67 (1H, m, CH₂a), 1.41-1.32 (2H, m, CH₂), 1.29-1.19 (3H, m, CH₂b and CH₂). ¹³C NMR (151 MHz, MeOD): δ 157.9 (C-6), 147.6 (C-2), 140.2 vinyl-C, 130.5 (C-9), 120.0 (vinyl-C), 118.9 (C-7), 112.6 (C-5), 109.9 (C-8), 86.3 (C-4′), 78.4 (C-1′), 75.2 (C-2′), 73.2 (C-3′), 63.7 (C-5′), 42.9 (CH), 34.2 (CH₂×2), 27.2 (CH₂), 27.1 (CH₂×2). HRMS (ESI⁺): m/z, [M+H]⁺ calcd for C₁₉H₂₇N₄O₄⁺, 375.2026; found, 375.2024.

### 7-((E)-pent-1-en-1-yl)-4-aza-7,9-dideazaadenosine (4.2m)

An argon-purged mixture of 4.8 (50 mg, 0.127 mmol), (E)-1-pentenylboronic acid (22 mg, 0.190 mmol), Na₂CO₃ (30 mg, 0.285 mmol), TPPTS (7.2 mg, 0.012 mmol) and Pd(OAc)₂ (1.5 mg, 0.006 mmol) in H₂O/MeCN (2/1, 3 mL) was heated to 100° C. and stirred for 1.5 h at the same temperature. The mixture was neutralized with 1N HCl(aq) and concentrated in vacuo. The residue was purified by column chromatography (silica gel, CH₂Cl₂/MeOH=15/1, 10/1) to afford 4.2m (27 mg, 63%). ¹H NMR (300 MHz, MeOD): δ 7.68 (1H, s, H-2), 6.88 (1H, s, H-8), 6.73 (1H, d, J=14.7 Hz, vinyl-CH), 6.14 (1H, dt, J₁=14.9 Hz, J₂=7.1 Hz, vinyl-CH), 5.23 (1H, d, J=6.5 Hz, H-1′), 4.46 (1H, t, J=5.9 Hz, H-2′), 4.17 (1H, t, J=4.8 Hz, H-3′), 4.03 (1H, q, J=3.8 Hz, H-4′), 3.81 (1H, dd, J₁=12.1 Hz, J₂=3.2 Hz, H-5′a), 3.69 (1H, dd, J₁=12.1 Hz, J₂=3.9 Hz, H-5′b), 2.23 (2H, q, J=3.8 Hz CH₂), 1.57-1.44 (2H, m, CH₂), 0.97 (3H, t, J=7.4 Hz, CH₃). ¹³C NMR (75 MHz, MeOD): δ 157.8 (C-6), 147.6 (C-2), 134.1 (vinyl-C), 130.4 (C-9), 122.4 (vinyl-C), 118.8 (C-7), 112.5 (C-5), 109.9 (C-8), 86.3 (C-4′), 78.4 (C-1′), 75.1 (C-2′), 73.2 (C-3′), 63.7 (C-5′), 36.2 (CH₂), 23.8 (CH₂), 14.1 (CH₃). HRMS (ESI⁺): m/z, [M+H]⁺ calcd for C₁₆H₂₃N₄O₄⁺, 335.1713; found, 335.1711.

### REFERENCES

1. Wu et al. Nature 2020, 579 (7798), 265-269.
2. Zhu et al. N. Engl. J. Med. 2020, 382 (8), 727-733.
3. Gorbalenya et al. Nat. Microbiol. 2020, 5 (4), 536-544.
4. Bian et al. Expert Rev. Vaccines 2021, 20 (4), 365-373.
5. Wang & Guan Med. Res. Rev. 2021, 41 (1), 5-28.
6. Pan et al. N. Engl. J. Med. 2021, 384 (6), 497-511.
7. Martinez Front. Immunol. 2021, 12, 635371.
8. Jonsson et al. Curr. Opin. Virol. 2021, 50, 1-7.
9. Wu et al. Cell Host Microbe 2020, 27 (3), 325-328.
10. Gordon et al. Nature 2020, 583 (7816), 459-468.
11. Jang et al. Proc. Natl. Acad. Sci. U. S. A. 2021, 118 (30).
12. Chen et al. Proc. Natl. Acad. Sci. U.S.A. 2009, 106 (9), 3484-3489.
13. Decroly et al. Nat. Rev. Microbiol. 2011, 10 (1), 51-65.
14. Chen & Guo Virol. Sin. 2016, 31 (1), 3-11.
15. Bobileva et al. ACS Med. Chem. Lett. 2021, 12 (7), 1102-1107.
16. Kasprzyk et al. Antiviral Res. 2021, 193, 105142.
17. Devkota, et al. bioRxiv 2021.
19. Patent application WO2015148746A1.
20. Patent application WO2011150356A1.
21. Metobo et al. Tetrahedron Lett. 2012, 53 (5), 484-486.
22. Western et al. J. Org. Chem. 2003, 68, 6767-6774.
23. Čapek et al. Org. Biomol. Chem. 2006, 4 (11), 2278-2284.
24. Wang et al. Tetrahedron Lett. 2005, 46 (50), 8777-8780.
25. Zhang et al. Angew. Chem. Int. Ed. 2019, 58 (32), 11049-11053.

1. A compound with general formula (I), or a stereoisomer, tautomer, racemic, salt, hydrate, N-oxide form, or solvate or prodrug thereof; 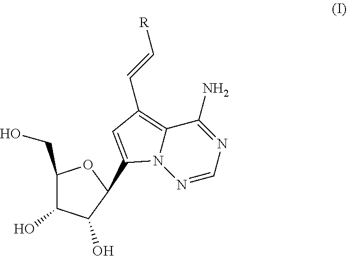
wherein R is selected from the group consisting of hydrogen, C₁₋₆alkyl, C₃₋₁₀cycloalkyl, C₆₋₁₀aryl, C₆₋₁₀arylC₁₋₆alkyl, heterocyclyl and heteroaryl, wherein said C₁₋₆alkyl, C₃₋₁₀cycloalkyl, C₆₋₁₀aryl, C₆₋₁₀aryl C₁₋₆alkyl, heterocyclyl and heteroaryl may be unsubstituted or substituted with halo, C₁₋₆alkyl, heteroC₁₋₆alkyl, C₁₋₆alkyloxy, hetero C₁₋₆alkyloxy, hydroxyl, amino, mono C₁₋₆alkylamino, diC₁₋₆alkylamino, C₆₋₁₀aryl, carboxy, aminocarbonyl, nitro, and cyano.

2. The compound of claim 1, wherein R is selected from the group consisting of C₁₋₄alkyl, C₃₋₈cycloalkyl, C₆₋₁₀aryl, C₆₋₁₀arylC₁₋₄alkyl, heterocyclyl, and heteroaryl.

3. The compound claim 1, wherein R is selected from the group consisting of hydrogen, methyl, ethyl, phenyl, p-methylphenyl, p-ethylphenyl, and p-propylphenyl.

4. The compound of claim 1, wherein R is hydrogen or p-methylphenyl.

5. The compound of claim 1, wherein the compound or a pharmaceutically acceptable salt thereof and at least one pharmaceutical acceptable carrier are comprised in a pharmaceutical.

6. (canceled)

7. A method for the treatment of a viral infection in a subject, the method comprising: administering to the subject the compound of claim 1.

8. The method according to claim 7, wherein the viral infection is an infection by an RNA virus.

9. The method according to claim 8, wherein the RNA virus is selected from the group consisting of coronavirus, measles, tacaribe virus, yellow fever virus, influenzavirus, Chikungunya, dengue, respiratory syncytial virus (RSV), human immunodeficiency virus (HIV), and norovirus.

10. The method according to claim 9, wherein the coronavirus is SARS-CoV-2.

11. A process for the preparation of a compound of formula (I) or a stereoisomer, tautomer, racemic, salt, hydrate, N-oxide form, or solvate or prodrug thereof 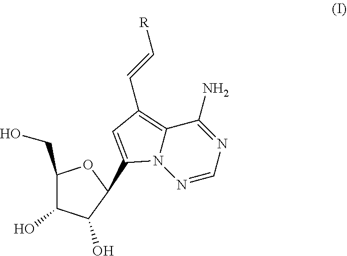
wherein R is selected from the group consisting of hydrogen, C₁₋₆alkyl, C₃₋₁₀cycloalkyl, C₆₋₁₀aryl, C₆₋₁₀arylC₁₋₆alkyl, heterocyclyl and heteroaryl, wherein said C₁₋₆alkyl, C₃₋₁₀cycloalkyl, C₆₋₁₀aryl, C₆₋₁₀aryl C₁₋₆alkyl, heterocyclyl and heteroaryl may be unsubstituted or substituted with halo, C₁₋₆alkyl, heteroC₁₋₆alkyl, C₁₋₆alkyloxy, hetero C₁₋₆alkyloxy, hydroxyl, amino, mono C₁₋₆alkylamino, diC₁₋₆alkylamino, C₆₋₁₀aryl, carboxy, aminocarbonyl, nitro, and cyano; the method comprising:
a) halogenating of a compound of formula (A) to produce a compound of formula (B), wherein X is I, Br, Cl or F; and 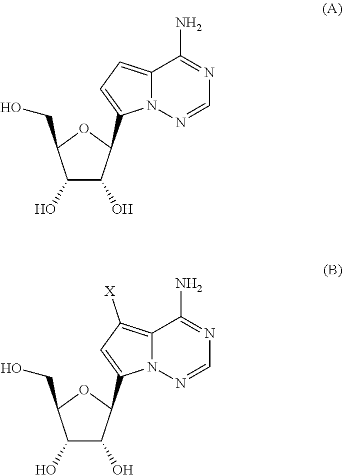
b) contacting a compound of formula (B) with a compound of formula (C) to produce the compound of formula (I).

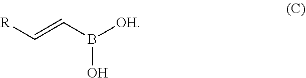

12. The process according to claim 11, wherein step b) is performed in the presence of a palladium catalyst.
